(19) United States
(12) Patent Application Publication
(10) Pub. No.: US 2026/0139282 A1
(43) Pub. Date: May 21, 2026

(54) LACTONE COMPOSITIONS AND METHODS OF MAKING THE SAME

(71) Applicant: Safion Renewables Inc., Louisville, CO (US)

(72) Inventors: Eric M. Karp, Arvada, CO (US); Jacob Scott Kruger, Arvada, CO (US); Ryan Prestangen, Denver, CO (US); Lauren Ann Riley Drumm, Lafayette, CO (US); Andrew John Borchert, Arvada, CO (US); Julia Rae Friedman, Boulder, CO (US); Katherine Noon, Westminster, CO (US); Sanket Gandhi, Columbus, GA (US); Alexander Hunt Jenkins, Louisville, CO (US)

(21) Appl. No.: 19/392,841

(22) Filed: Nov. 18, 2025

Related U.S. Application Data
(60) Provisional application No. 63/721,905, filed on Nov. 18, 2024.

(51) Int. Cl. C12P 7/6463; C12N 1/00; C12N 1/16; C12R 1/645

(52) U.S. Cl. CPC C12P 7/6463; C12N 1/005; C12N 1/16; C12R 2001/645

(57) ABSTRACT

Provided herein are methods of making compositions comprising mixtures of lactones. Also provided herein are mixtures of lactones produced by the methods provided herein. An example lactone composition can include a 5-hydroxy-2-decenoic acid-δ-lactone, a 3,5-dihydroxydecanoic acid-δ-lactone, and sulfur, nitrogen, phosphorus, potassium, magnesium, calcium, sodium, iron, aluminum, silicon, organic chloride, 3,5-dihydroxydecanoic acid, a carbohydrate, free fatty acids, or a combination thereof.

### CROSS-REFERENCE TO RELATED APPLICATION

This application claims the benefit of priority to U.S. Provisional Patent Ser. No. 63/721,905 entitled “LACTONE COMPOSITIONS AND METHODS OF MAKING THE SAME,” filed Nov. 18, 2024, the disclosure of which is incorporated herein in its entirety by reference.

### BACKGROUND

Distillation, acidification, and saponification may be used alone or in combination to produce desirable chemical products from oils. Cyclic carboxylic esters, also known as lactones, are naturally occurring compounds that often contribute to the flavor and fragrance of fruits and dairy products. Massoia lactone, otherwise known as 5-hydroxy-2-decenoic acid-δ-lactone and named for the bark of the Massoia tree (Cryptocarya massoia), is a lactone commonly utilized in the food and beverage industries, as well as in perfumery, for its creamy, coconut-like odor and flavor. Massoia lactone has also been studied for its antifungal properties.

### SUMMARY

In an example, a composition can include (a) a 5-hydroxy-2-decenoic acid-6-lactone; (b) a 3,5-dihydroxydecanoic acid-δ-lactone; and (c) sulfur, nitrogen, phosphorus, potassium, magnesium, calcium, sodium, iron, aluminum, zinc, manganese, copper, silicon, organic chlorides, 3,5-dihydroxydecanoic acid, a carbohydrate, free fatty acids, or a combination thereof.

In an example, a method can include (a) harvesting a bioproduct produced by an engineered organism, the bioproduct comprising one or more lipids, the one or more lipids comprising a carbohydrate and 1 to 5 C₈-C₁₆ polyester tails polyester tails; and (b) distilling the bioproduct, wherein the distilling produces a composition comprising one or more substituted or unsubstituted lactones.

In an example, a method can include (a) Harvesting a bioproduct produced by an engineered organism, the bioproduct comprising one or more lipids, the one or more lipids comprising a carbohydrate and 1 to 5 C₈-C₁₆ polyester tails; (b) contacting at least a portion of the one or more lipids with a base or with an acid catalyst; and (c) distilling the bioproduct, wherein the distilling produces a composition comprising one or more substituted or unsubstituted lactones, or a combination thereof.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 shows the relative abundance of various lipids as described herein in an example.

FIG. 2 shows estimated hydrocarbon mole percentages of lipids as described herein in an example.

FIG. 3 depicts saponification yield vs time for various concentration and quantity of base in an example.

FIG. 4 shows concentrations of lactones versus reaction time for mixtures with various pH in an example.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Provided herein are compositions and methods for producing compositions comprising one or more substituted or unsubstituted (e.g., hydroxylated or unhydroxylated, saturated or unsaturated) lactones, such as a 5-hydroxy-2-decenoic acid-δ-lactone or a 3,5-dihydroxydecanoic acid-δ-lactone. Provided herein are methods of preparing substituted or unsubstituted lactones comprising the use of a bio-derived heavy oil (e.g., lipids described herein) that may be sustainable and comprise a low carbon intensity (CI). The substituted or unsubstituted lactones provided herein may be used to impart coconut flavor and/or aroma (e.g. as a Massoia bark essential oil replacement) to cosmetic products, foods and beverages, and perfumes. The substituted or unsubstituted lactones provided herein may also be used as an antifungal.

More specifically, discussed herein are lactone compositions and sustainable methods for their production as alternatives to naturally-derived Massoia lactone. Compositions can include 5-hydroxy-2-decenoic acid-δ-lactone and 3,5-dihydroxydecanoic acid-δ-lactone, along with various trace components including sulfur, nitrogen, phosphorus, metals, and free fatty acids. Processes can involve harvesting bio-derived lipids from engineered microorganisms (e.g., Aureobasidium pullulans) that contain carbohydrate headgroups with 1 to 5 C₈-C₁₆ polyester tails, followed by treatment through saponification with bases (e.g., sodium hydroxide or potassium hydroxide), optional acidification with mineral acids, and distillation at temperatures ranging from 100° C. to over 250° C. depending on the specific process route.

The resulting lactone mixtures can exhibit coconut-like aroma and flavor characteristics, making them suitable for use in food products and cosmetics as sustainable replacements for Massoia essential oil, while offering the advantage of lower carbon intensity compared to tree bark extraction methods. Discussed herein are multiple processing pathways including continuous production methods and achieves lactone yields of at least 30-95% by weight through various combinations of chemical treatment and thermal distillation processes.

The processes and compositions discussed herein address the expensive and destructive nature of extracting Massoia lactone from Massoia tree bark. Current extraction methods require harvesting bark from Massoia trees, which creates both economic and environmental challenges including high costs, environmental destruction through tree harvesting, and supply chain constraints that limit sustainable large-scale commercial production of this important flavor and fragrance compound with coconut-like odor and flavor characteristics commonly used in food, beverages, and perfumery.

The processes discussed herein provide lactone production through a biotechnology-based production system using genetically engineered Aureobasidium pullulans that produces specific bio-derived lipid precursors with defined structural features (e.g., a carbohydrate headgroup with 1 to 5 C₈-C₁₆ polyester tails). The engineered organism incorporates targeted modifications to genes related to polymalic acid production, pullulan production, oxygen binding hemoglobin, and pathway activator genes to optimize lipid substrate production at an industrial scale. This can provide multiple integrated processing routes including saponification+acidification+distillation (Route A), direct acidification+distillation (Route B), and saponification+thermal lactonization+distillation (Route C), enabling flexible manufacturing approaches to convert the bio-derived lipids into the target lactones.

This can achieve superior environmental and economic performance by delivering significantly lower carbon intensity (CI) with lactones having CI of less than 1 kg CO₂e/kg of lactone and potentially negative CI in some cases, while enabling continuous production processes and industrial scalability (e.g., up to 1000+ kg/h production capacity). The bio-derived lactone compositions can maintain the same coconut-like odor and flavor characteristics as naturally-derived Massoia lactone, making them direct replacements for Massoia essential oil in food products and cosmetics, thus addressing both the sustainability challenge and supply limitation issues while preserving product performance.

### Certain Definitions

As used herein and in the appended claims, the singular forms “a,” “and,” and “the” include plural referents unless the context clearly dictates otherwise. Thus, for example, reference to “an agent” includes a plurality of such agents and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

As used herein, the term “heavy oil,” and grammatical variants thereof, refers generally to a mixture of lipids described by Formula (I), and shown in Table 1. These lipids are generally molecules within a subclass of biosurfactant glycolipids identified as polyol lipids that can be produced by yeast-like fungi of the Aureobasidium genus. Heavy oils are amphiphilic molecules comprising a polyol head group such as mannitol, arabitol, or glycerol, attached to one or multiple O-acylated 3,5-dihydroxydecanoic acid or 3,5-dihydroxydodecanoic acid group tails. The tails may be acetylated at the hydroxyl (—OH) group. 3,5-dihydroxydecanoic acid derived heavy oils lacking the polyol head group are classified as aglycone oligo-dihydroxydecanoic acids (DDA) (formerly exophilins).

Carbon Intensity (CI), as described herein, can refer to a measure of carbon dioxide and other greenhouse gases per unit of activity (e.g., manufacture of a composition provided herein). In some embodiments, CI is measured using the Greenhouse gases, Regulated Emissions, and Energy use in Technologies (GREET) model, which uses chemical, feedstock, process, utility and other relevant input data related to the production of a fuel or chemical to calculate the emissions associated with that fuel or chemical and determine a CI score for that product based upon the total greenhouse gas emissions produced per unit of activity (e.g., as described at http://greet.anl.gov/). In some embodiments, CI is calculated using the R&D GREET 2024 model as described at http://greet.anl.gov/.

“Amino” refers to the —NH2 functional group.

“Hydroxyl” refers to the —OH functional group.

“O-acylation” as used herein refers to the attachment of an acyl group (R—C═O) to an oxygen atom in a molecule such as to form an ester (R—C(═O)O—R′).

“Alkyl” refers to a straight-chain, or branched-chain saturated hydrocarbon monoradical. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀ alkyl. In some embodiments, the alkyl is a C₁₋₆ alkyl. In some embodiments, the alkyl is a C₁₋₅ alkyl. In some embodiments, the alkyl is a C₁₋₄ alkyl. In some embodiments, the alkyl is a C₁₋₃ alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe.

“Continuous production process” or “continuous process” refer to processes which are designed to manufacture products constantly without interruption. A continuous production process or continuous process may occur in a single fermenter or reaction vessel, or in the separation of the oil from the other fermenter contents, or in the oil purification process, or in the lactone production process, or in the lactone purification process, or in any combination of process steps.

The term “modification” with respect to a gene or a nucleic acid (and related terms such as “modify” or “modified”) can mean knockout, disruption, truncation, knockdown, inhibition, insertion, deletion, mutation, or substitution. It can result in an increase or enhancement in expression of the modified gene. It can result in a decrease in expression of the modified gene.

### Lactone Compositions

Provided herein, in some embodiments, are compositions comprising one or more substituted or unsubstituted (e.g., saturated or unsaturated) lactones, such as a 5-hydroxy-2-decenoic acid-δ-lactone or a 3,5-dihydroxydecanoic acid-δ-lactone. In some embodiments, provided herein are compositions comprising one or more substituted or unsubstituted (e.g., saturated or unsaturated) lactones. In some embodiments, the substituted or unsubstituted (e.g., saturated or unsaturated) lactone is a 5-hydroxy-2-decenoic acid-6-lactone. In some embodiments, the substituted or unsubstituted (e.g., saturated or unsaturated) lactone is a 3,5-dihydroxydecanoic acid-δ-lactone.

In some embodiments, the one or more substituted or unsubstituted (e.g., saturated or unsaturated) lactones comprise 5-hydroxy-2-decenoic acid-δ-lactone (i.e. Massoia lactone):

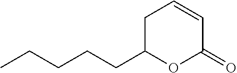

In some embodiments, the 5-hydroxy-2-decenoic acid-δ-lactone is (R)-5,6-dihydro-6-pentyl-2H-pyran-2-one.

In some embodiments, the one or more substituted or unsubstituted (e.g., saturated or unsaturated) lactones comprise 3,5-dihydroxydecanoic acid-δ-lactone:

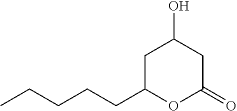

In some embodiments, the one or more substituted or unsubstituted (e.g., saturated or unsaturated) lactones comprise 5-hydroxy-2-decenoic acid-δ-lactone and 3,5-dihydroxydecanoic acid-δ-lactone. In some embodiments, the one or more substituted or unsubstituted (e.g., saturated or unsaturated) lactones comprise 5-hydroxy-2-decenoic acid-δ-lactone and 3,5-dihydroxydecanoic acid-δ-lactone at a ratio of at least 10:1, 9:1, 8:1, 7:1, 6:1, 5:1, or 4:1. In some embodiments, the one or more substituted or unsubstituted (e.g., saturated or unsaturated) lactones comprise 5-hydroxy-2-decenoic acid-δ-lactone and 3,5-dihydroxydecanoic acid-δ-lactone at a ratio of at most 1:10, 1:9, 1:8, 1:7, 1:6, 1:5, or 1:4. In some embodiments, the one or more substituted or unsubstituted (e.g., saturated or unsaturated) lactones comprise 5-hydroxy-2-decenoic acid-δ-lactone and 3,5-dihydroxydecanoic acid-δ-lactone at a ratio of about 4:1, 3:1, 2:1, 1:1, 1:2, 1:3, or about 1:4. In some embodiments, the one or more substituted or unsubstituted (e.g., saturated or unsaturated) lactones comprise 5-hydroxy-2-decenoic acid-δ-lactone and 3,5-dihydroxydecanoic acid-δ-lactone at a ratio of from about 10:1 to about 1:10, from about 5:1 to about 1:5, or from about 2:1 to about 1:2.

In some embodiments, the composition comprises the substituted or unsubstituted (e.g., saturated or unsaturated) lactone in an amount of at least 40 wt %. In some embodiments, the composition comprises the substituted or unsubstituted (e.g., saturated or unsaturated) lactone in an amount of at least 60 wt %. In some embodiments, the composition comprises the substituted or unsubstituted (e.g., saturated or unsaturated) lactone in an amount of at least about 20 wt %, about 30 wt %, about 40 wt %, about 50 wt %, about 60 wt %, about 70 wt %, about 75 wt %, about 80 wt %, about 85 wt %, about 90 wt %, or about 95 wt %. In some embodiments, the composition comprises the substituted or unsubstituted (e.g., saturated or unsaturated) lactone in an amount of at most about 95 wt %, about 90 wt %, about 85 wt %, about 80 wt %, about 75 wt %, about 70 wt %, about 60 wt %, about 50 wt %, about 40 wt %, about 30 wt %, or about 20 wt %. In some embodiments, the composition comprises the substituted or unsubstituted (e.g., saturated or unsaturated) lactone in an amount of from about 20 wt % to about 95 wt %, about 30 wt % to about 90 wt %, about 30 wt % to about 85 wt %, about 40 wt % to about 85 wt %, about 20 wt % to about 75 wt %, about 40 wt % to about 75 wt %, or about 50 wt % to about 80 wt %. In some embodiments, the composition comprises the substituted or unsubstituted (e.g., saturated or unsaturated) lactone in an amount of about 20 wt %, about 30 wt %, about 40 wt %, about 50 wt %, about 60 wt %, about 70 wt %, about 75 wt %, about 80 wt %, about 85 wt %, about 90 wt %, or about 95 wt %.

In some embodiments, the composition comprises the 5-hydroxy-2-decenoic acid-δ-lactone and/or 3,5-dihydroxydecanoic acid-δ-lactone in an amount of at least 40 wt %. In some embodiments, the composition comprises the 5-hydroxy-2-decenoic acid-δ-lactone and/or 3,5-dihydroxydecanoic acid-δ-lactone in an amount of at least 60 wt %. In some embodiments, the composition comprises the 5-hydroxy-2-decenoic acid-δ-lactone and/or 3,5-dihydroxydecanoic acid-δ-lactone in an amount of at least about 20 wt %, about 30 wt %, about 40 wt %, about 50 wt %, about 60 wt %, about 70 wt %, about 75 wt %, about 80 wt %, about 85 wt %, about 90 wt %, or about 95 wt %. In some embodiments, the composition comprises the 5-hydroxy-2-decenoic acid-δ-lactone and/or 3,5-dihydroxydecanoic acid-δ-lactone in an amount of at most about 95 wt %, about 90 wt %, about 85 wt %, about 80 wt %, about 75 wt %, about 70 wt %, about 60 wt %, about 50 wt %, about 40 wt %, about 30 wt %, or about 20 wt %. In some embodiments, the composition comprises the 5-hydroxy-2-decenoic acid-δ-lactone and/or 3,5-dihydroxydecanoic acid-δ-lactone in an amount of from about 20 wt % to about 95 wt %, about 30 wt % to about 90 wt %, about 30 wt % to about 85 wt %, about 40 wt % to about 85 wt %, about 20 wt % to about 75 wt %, about 40 wt % to about 75 wt %, or about 50 wt % to about 80 wt %. In some embodiments, the composition comprises the 5-hydroxy-2-decenoic acid-δ-lactone and/or 3,5-dihydroxydecanoic acid-δ-lactone in an amount of about 20 wt %, about 30 wt %, about 40 wt %, about 50 wt %, about 60 wt %, about 70 wt %, about 75 wt %, about 80 wt %, about 85 wt %, about 90 wt %, or about 95 wt %.

In some embodiments, a variety of one or more impurities may be present, such as 0.0 to 1.0 wt % hexanal, 0.0 to 1.0 wt % 4,4a,5,6,7,8a-h exahydropyrano[2,3-b]pyran, 0.0 to 1.0 wt % 2H,7H-pyrano[2,3-b]pyran, 0.0 to 5.0 wt % 2-methyl-3-heptyn-2-ol, 0.0 to 5.0 wt % 2,6-dimethyl-4-hepten-3-one, or combinations thereof.

In some embodiments, the compositions comprising one or more substituted or unsubstituted lactones further comprise sulfur, nitrogen, phosphorus, potassium, magnesium, calcium, sodium, iron, aluminum, zinc, manganese, copper, silicon, organic chlorides, 3,5-dihydroxydecanoic acid, 3,5-dihydroxydodecanoic acid, a carbohydrate, or free fatty acids. In some embodiments, the compositions comprise sulfur, nitrogen, phosphor us, potassium, magnesium, calcium, sodium, iron, aluminum, zinc, manganese, copper, silicon, organic chlorides, 3,5-dihydroxydecanoic acid, 3,5-dihydroxydodecanoic acid, a carbohydrate, free fatty acids, or a combination thereof. In some embodiments, the compositions comprise water. In some embodiments, the compositions comprise sulfur. In some embodiments, the compositions comprise nitrogen. In some embodiments, the compositions comprise phosphorus. In some embodiments, the compositions comprise potassium. In some embodiments, the compositions comprise magnesium. In some embodiments, the compositions comprise calcium. In some embodiments, the compositions comprise sodium. In some embodiments, the compositions comprise iron. In some embodiments, the compositions comprise aluminum. In some embodiments, the compositions comprise zinc. In some embodiments, the compositions comprise manganese. In some embodiments, the compositions comprise copper. In some embodiments, the compositions comprise silicon. In some embodiments, the compositions comprise organic chlorides. In some embodiments, the compositions comprise 3,5-dihydroxydecanoic acid. In some embodiments, the compositions comprise 3,5-dihydroxydodecanoic acid. In some embodiments, the compositions comprise a carbohydrate. In some embodiments, the carbohydrate comprises mannitol, arabitol, glycerol, or a combination thereof. In some embodiments, the compositions comprise free fatty acids. In some embodiments, the carbohydrate comprises mannitol. In some embodiments, the compositions comprise cell debris.

In some embodiments, the composition comprises at least about 1 wt %, 2 wt %, 3 wt %, 4 wt %, or 5 wt % water. In some embodiments, the composition comprises about 5 wt %, 4 wt %, 3 wt %, 2 wt %, 1 wt %, or less, water. In some embodiments, the composition comprises between at least about 0 wt % and 5 wt % water.

In some embodiments, the composition comprises at least about 1, 2, 3, 4, 5, 6, 7, 8, 9, 10, 11, 12, 13, 14, 15, 16, 17, 18, 19, 20, 30, 40, 50, 60, 70, 80, 90, 100, or more, ppm sulfur. In some embodiments, the composition comprises about 100, 90, 80, 70, 60, 50, 40, 30, 20, 19, 18, 17, 16, 15, 14, 13, 12, 11, 10, 9, 8, 7, 6, 5, 4, 3, 2, 1, or less, ppm sulfur. In some embodiments, the composition comprises between at least about 1 and 100 ppm sulfur.

In some embodiments, the composition comprises at least about 10, 20, 30, 40, 50, 60, 70, 71, 72, 73, 74, 75, 76, 77, 78, 79, 80, 81, 82, 83, 84, 85, 86, 87, 88, 89, 90, 100, 110, 120, 130, 140, 150, 160, 170, 180, 190, 200, or more, ppm nitrogen. In some embodiments, the composition comprises about 200, 190, 180, 170, 160, 150, 140, 130, 120, 110, 100, 90, 89, 88, 87, 86, 85, 84, 83, 82, 81, 80, 79, 78, 77, 76, 75, 74, 73, 72, 71, 70, 60, 50, 40, 30, 20, 10, or less, ppm nitrogen. In some embodiments, the composition comprises between at least about 10 and 200 ppm nitrogen.

In some embodiments, the composition comprises at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, or more, ppm phosphorus. In some embodiments, the composition comprises about 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or less, ppm phosphorus. In some embodiments, the composition comprises between at least about 0.01 and 10 ppm phosphorus.

In some embodiments, the composition comprises at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, or more, ppm potassium. In some embodiments, the composition comprises about 10, 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or less, ppm potassium. In some embodiments, the composition comprises between at least about 0.01 and 10 ppm potassium.

In some embodiments, the composition comprises at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, or more, ppm magnesium. In some embodiments, the composition comprises about 10, 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or less, ppm magnesium. In some embodiments, the composition comprises between at least about 0.01 and 10 ppm magnesium.

In some embodiments, the composition comprises at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 5, 6, 7, 8, 9, 10, or more, ppm calcium. In some embodiments, the composition comprises about 10, 9, 8, 7, 6, 5, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, or less, ppm calcium. In some embodiments, the composition comprises between at least about 0.1 and 10 ppm calcium.

In some embodiments, the composition comprises at least about 1, 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 8, 9, 10, or more, ppm sodium. In some embodiments, the composition comprises about 10, 9, 8, 7, 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4, 3, 2, 1, or less, ppm sodium. In some embodiments, the composition comprises between at least about 1 and 10 ppm sodium.

In some embodiments, the composition comprises at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, or more, ppm iron. In some embodiments, the composition comprises about 10, 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or less, ppm iron. In some embodiments, the composition comprises between at least about 0.01 and 10 ppm iron.

In some embodiments, the composition comprises at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, or more, ppm aluminum. In some embodiments, the composition comprises about 10, 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or less, ppm aluminum. In some embodiments, the composition comprises between at least about 0.01 and 10 ppm aluminum.

In some embodiments, the composition comprises at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, or more, ppm zinc. In some embodiments, the composition comprises about 10, 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or less, ppm zinc. In some embodiments, the composition comprises between at least about 0.01 and 10 ppm zinc.

In some embodiments, the composition comprises at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, or more, ppm manganese. In some embodiments, the composition comprises about 10, 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or less, ppm manganese. In some embodiments, the composition comprises between at least about 0.01 and 10 ppm manganese.

In some embodiments, the composition comprises at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, or more, ppm copper. In some embodiments, the composition comprises about 10, 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or less, ppm copper. In some embodiments, the composition comprises between at least about 0.01 and 10 ppm copper.

In some embodiments, the composition comprises at least about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 5, 6, 7, 8, 9, 10, or more, ppm silicon. In some embodiments, the composition comprises about 10, 9, 8, 7, 6, 5, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, or less, ppm silicon. In some embodiments, the composition comprises between at least about 1 and 10 ppm silicon.

In some embodiments, the composition comprises at least about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 4, 5, 6, 7, 8, 9, 10, or more, ppm organic chlorides. In some embodiments, the composition comprises about 10, 9, 8, 7, 6, 5, 4, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, or less, ppm organic chlorides. In some embodiments, the composition comprises between at least about 1 and 10 ppm organic chlorides.

In some embodiments, the composition comprises at least about 1, 2, 3, 4, 5, 6, 7, 8, 9, 10, 11, 12, 13, 14, 15, 16, 17, 18, 19, 20, 30, 40, 50, 60, 70, 80, 90, 100, or more, ppm 3,5-dihydroxydodecanoic acid. In some embodiments, the composition comprises about 100, 90, 80, 70, 60, 50, 40, 30, 20, 19, 18, 17, 16, 15, 14, 13, 12, 11, 10, 9, 8, 7, 6, 5, 4, 3, 2, 1, or less, ppm 3,5-dihydroxydodecanoic acid. In some embodiments, the composition comprises between at least about 1 and 100 ppm 3,5-dihydroxydodecanoic acid.

In some embodiments, a free carbohydrate (e.g., mannitol) can be present in an amount of about 0.0 to 1.0 wt %, such as about 0.0, 0.1, 0.2, 0.3, 0.4, 0.5, 0.6, 0.7, 0.8, 0.9, or 1.0 wt %.

In some embodiments, free fatty acids can be present in an amount of about 0.0 to 1.0 wt %, such as about 0.0, 0.1, 0.2, 0.3, 0.4, 0.5, 0.6, 0.7, 0.8, 0.9, or 1.0 wt %.

In some embodiments, the free fatty acids comprise C22:0 (i.e., behenic acid), C18:3 (i.e., linolenic acid), C24:0 (i.e., lignoceric acid), C18:1 (i.e., oleic acid), or C14:0 (i.e., myristic acid). In some embodiments, the free fatty acids may comprise at least 50% C22:0. In some embodiments, the free fatty acids comprise at least 20% C18:3. In some embodiments, the free fatty acids comprise at least 10% C24:0. In some embodiments, the free fatty acids comprise at least 1% C18:1. In some embodiments, the free fatty acids comprise or at least 0.1% C14:0.

In some embodiments, the free fatty acids comprise at least 52% C22:0, at least 21% C18:3, at least 17% C24:0, at least 5% C18:1, or at least 2% C14:0. In some embodiments, the free fatty acids comprise at least 52% C22:0. In some embodiments, the free fatty acids comprise at least 21% C18:3. In some embodiments, the free fatty acids comprise at least 17% C24:0. In some embodiments, the free fatty acids comprise at least 5% C18:1. In some embodiments, the free fatty acids comprise at least at least 2% C14:0.

In some embodiments, the free fatty acids comprise at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, or more, C22:0 (i.e., behenic acid). In some embodiments, the free fatty acids comprise about 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, or less, C22:0 (i.e, behenic acid). In some embodiments, the free fatty acids comprise between at least about 15% and 75% C22:0 (i.e., behenic acid).

In some embodiments, the free fatty acids comprise at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or more, C18:3 (i.e., linolenic acid). In some embodiments, the free fatty acids comprise about 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less, C18:3 (i.e., linolenic acid). In some embodiments, the free fatty acids comprise between at least about 1% and 40% C18:3 (i.e., linolenic acid).

In some embodiments, the free fatty acids comprise at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or more, C24:0 (i.e., lignoceric acid). In some embodiments, the free fatty acids comprise about 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less, C24:0 (i.e., lignoceric acid). In some embodiments, the free fatty acids comprise between at least about 1% and 40% C24:0 (i.e., lignoceric acid).

In some embodiments, the free fatty acids comprise at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or more, C18:1 (i.e., oleic acid). In some embodiments, the free fatty acids comprise about 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 4%1, 13%1, 12%, 1%, 10%, 9%, 8%, 7%, 6%, 5%, 4%, 3%, 2%, 1%, or less, C18:1 (i.e., oleic acid). In some embodiments, the free fatty acids comprise between at least about 1% and 40% C18:1 (i.e., oleic acid).

In some embodiments, the free fatty acids comprise at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, or more, C14:0 (i.e., myristic acid). In some embodiments, the free fatty acids comprise about 30%, 29%, 28%, 27%, 26%, 25%, 24%, 23%, 22%, 21%, 20%, 19%, 18%, 17%, 16%, 1, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or less, C14:0 (i.e., myristic acid). In some embodiments, the free fatty acids comprise between at least about 0.1% and 30% C14:0 (i.e., myristic acid).

In some embodiments, the composition includes one or more impurities, such as hexanal, 4,4a,5,6,7,8a-h exahydropyrano[2,3-b]pyran, 2H,7H-pyrano[2,3-b]pyran, 2-methyl-3-heptyn-2-ol, 0.0 to 1.0 wt % 4,4a,5,6,7,8a-h exahydropyrano[2,3-b]pyran, 2H,7H-pyrano[2,3-b]pyran, 2-methyl-3-heptyn-2-ol, or combinations thereof.

In some embodiments, hexanal can be present in an amount of about 0.0 to 1.0 wt %, such as about 0.0, 0.1, 0.2, 0.3, 0.4, 0.5, 0.6, 0.7, 0.8, 0.9, or 1.0 wt %.

In some embodiments, 4,4a,5,6,7,8a-h exahydropyrano[2,3-b]pyran can be present in an amount of about 0.0 to 1.0 wt %, such as about 0.0, 0.1, 0.2, 0.3, 0.4, 0.5, 0.6, 0.7, 0.8, 0.9, or 1.0 wt %.

In some embodiments, 2H,7H-pyrano[2,3-b]pyran can be present in an amount of about 0.0 to 1.0 wt %, such as about 0.0, 0.1, 0.2, 0.3, 0.4, 0.5, 0.6, 0.7, 0.8, 0.9, or 1.0 wt %.

In some embodiments, 2-methyl-3-heptyn-2-ol can be present in an amount of about 0.0 to 5.0 wt %, such as about 0.0, 0.5, 1.0, 1.5, 2.0, 2.5, 3.0, 3.5, 4.0, 4.5, or 5.0 Wt %.

In some embodiments, 2,6-dimethyl-4-hepten-3-one can be present in an amount of about 0.0 to 5.0 wt %, such as about 0.0, 0.5, 1.0, 1.5, 2.0, 2.5, 3.0, 3.5, 4.0, 4.5, or 5.0 wt %.

In some embodiments, the compositions comprise a base used to saponify the bioproduct or the one or more lipids. In some embodiments, the compositions comprise a catalyst used to treat the bioproduct or the one or more lipids. In some embodiments, the compositions comprise an acid catalyst used to acidify the bioproduct or the one or more lipids.

### Food and Cosmetic Products

At least one of the one or more substituted or unsubstituted lactones provided herein may have a coconut smell. At least one of the one or more substituted or unsubstituted lactones provided herein may have a coconut taste. At least one of the one or more substituted or unsubstituted lactones provided herein may comprise the chemical responsible for the coconut smell (e.g., fragrance) produced in the bark of the Massoia tree (Cryptocarya massoia). In some embodiments, the one or more substituted or unsubstituted lactones provided herein may be comprised in cosmetic products or food products. In some embodiments, the one or more substituted or unsubstituted lactones may be used as a Massoia essential oil replacement. In some embodiments, the one or more substituted or unsubstituted lactones may be used as an antifungal product.

In some embodiments, the compositions provided herein (e.g., the compositions comprising one or more lactones) may be comprised in a food product. In some instances, the compositions provided herein may be used as a Massoia essential oil replacement. In some embodiments, the compositions provided herein may be used as a coconut flavorant. In some embodiments, the compositions provided herein may be used as a coconut fragrance.

In some embodiments, the compositions provided herein (e.g., the compositions comprising one or more lactones) may be comprised in a cosmetic product. In some instances, the compositions provided herein may be used as a Massoia essential oil replacement. In some embodiments, the compositions provided herein may be used as a coconut flavorant. In some embodiments, the compositions provided herein may be used as a coconut fragrance.

In some embodiments, a cosmetic product comprising the compositions providing one or more lactones may be formulated as a topical skin composition. In some embodiments, a cosmetic product comprising the compositions providing one or more lactones may be included in a cosmetic vehicle. A cosmetic vehicle may be a cream, a lotion, a gel, an ointment, a serum, an emulsion (e.g., oil-in-water, water-in-oil, silicone-in-water, water-in-silicone, water-in-oil-in-water, oil-in-water-in-oil, oil-in-water-in-silicone, etc.), a solution (e.g., aqueous solution or hydro-alcoholic solution), an anhydrous base (e.g., a lipstick or a powder), a milk, a paste, an aerosol, a solid form, or a combination thereof. In some embodiments, a cosmetic product comprising the compositions providing one or more lactones may be formulated as a moisturizer, mask, foundation, freshener, cleanser, toner, or combination thereof.

### Methods of Making Lactones

Provided herein, in some embodiments, are methods of preparing one or more substituted or unsubstituted (e.g., saturated or unsaturated) lactones, such as a 5-hydroxy-2-decenoic acid-δ-lactone or a 3,5-dihydroxydecanoic acid-δ-lactone. In some embodiments, provided herein is a method of preparing one or more substituted or unsubstituted (e.g., saturated or unsaturated) lactones. In some embodiments, the substituted or unsubstituted (e.g., saturated or unsaturated) lactone is a 5-hydroxy-2-decenoic acid-δ-lactone. In some embodiments, the substituted or unsubstituted (e.g., saturated or unsaturated) lactone is a 3,5-dihydroxydecanoic acid-δ-lactone.

In some embodiments, the methods herein comprise providing a composition, wherein the composition comprises one or more lipids comprising a carbohydrate and 1 to 5 C5-C1₆ polyester tails.

### Bioproduct Starting Materials

In some embodiments, the methods comprise producing a composition comprising one or more substituted or unsubstituted lactones. In some embodiments, the lactones are saturated or unsaturated lactones. In some embodiments, the composition comprises one or more substituted or unsubstituted saturated or unsaturated lactones. In some embodiments, the composition comprises one or more substituted saturated or unsaturated lactones. In some embodiments, the composition comprises one or more saturated or unsaturated lactones substituted with one or more C₁-C₈ alkyl groups and/or hydroxyl groups.

In some embodiments, the methods comprise harvesting a bioproduct produced by an engineered organism. In some embodiments, the bioproduct comprises one or more lipids comprising a carbohydrate and 1 to 5 C₈-C₁₆ polyester tails. In some embodiments, the methods comprise providing a bioproduct or composition comprising one or more lipids comprising a carbohydrate and 1 to 5 C₈-C₁₆ polyester tails.

In some embodiments, the bioproduct or composition comprises one or more lipids comprising a carbohydrate substituted with between 1 to 5 C₈-C₁₆ polyester tails. In some embodiments, the bioproduct or composition comprises one or more lipids comprising mannitol, glycerol, or arabitol, substituted with between 1 to 5 C₈-C₁₆ polyester tails. In some embodiments, the bioproduct or composition comprise 3,5-dihydroxydecanoyl and/or 5-hydroxy-2-decenoyl esters of arabitol, mannitol, or glycerol.

In some embodiments, the one or more lipids are represented by a structure of Formula (I):

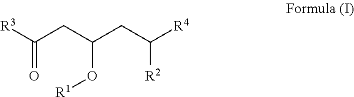

In some embodiments, R¹ is hydrogen (H) or acetyl (CH₃CO). In some embodiments, Riis hydrogen (H) or R′CO. In some embodiments, R′ is C₁-C₆ alkyl. In some embodiments, R′ is methyl. In some embodiments R¹ is hydrogen. In some embodiments, R¹ is acetyl (CH₃CO).

In some embodiments, R² is hydrogen. In some embodiments, R² is 1 to 10 O-acylated 3,5-dihydroxydecanoate. In some embodiments, R² is 1 to 8 O-acylated 3,5-dihydroxydecanoate. In some embodiments, R² is 1 to 6 O-acylated 3,5-dihydroxydecanoate. In some embodiments, R² is 1 to 5 O-acylated 3,5-dihydroxydecanoate. In some embodiments, R² is 1 to 4 O-acylated 3,5-dihydroxydecanoate. In some embodiments, R² is 1 to 3 O-acylated 3,5-dihydroxydecanoate. In some embodiments, R² is 1 O-acylated 3,5-dihydroxydecanoate. In some embodiments, R² is 2 O-acylated 3,5-dihydroxydecanoate. In some embodiments, R² is 3 O-acylated 3,5-dihydroxydecanoate. In some embodiments, R² is 4 O-acylated 3,5-dihydroxydecanoate. In some embodiments, R² is 5 O-acylated 3,5-dihydroxydecanoate. In some embodiments, R² is 6 O-acylated 3,5-dihydroxydecanoate. In some embodiments, R² is 7 O-acylated 3,5-dihydroxydecanoate. In some embodiments, R² is 8 O-acylated 3,5-dihydroxydecanoate. In some embodiments, R² is 9 O-acylated 3,5-dihydroxydecanoate. In some embodiments, R² is 10 O-acylated 3,5-dihydroxydecanoate.

In some embodiments, in R2, one or more O-acylated 3,5-dihydroxydecanoate may be substituted with O-acylated 3,5-dihydroxydodecanoate.

In some embodiments, R² is 1 to 10 O-acylated 3,5-dihydroxydecanoate, 3,5-dihydroxydodecanoate, or a combination thereof. In some embodiments, R² is 1 to 8 O-acylated 3,5-dihydroxydecanoate, 3,5-dihydroxydodecanoate, or a combination thereof. In some embodiments, R² is 1 to 6 O-acylated 3,5-dihydroxydecanoate, 3,5-dihydroxydodecanoate, or a combination thereof. In some embodiments, R² is 1 to 5 O-acylated 3,5-dihydroxydecanoate, 3,5-dihydroxydodecanoate, or a combination thereof. In some embodiments, R² is 1 to 4 O-acylated 3,5-dihydroxydecanoate, 3,5-dihydroxydodecanoate, or a combination thereof. In some embodiments, R² is 1 to 3 O-acylated 3,5-dihydroxydecanoate, 3,5-dihydroxydodecanoate, or a combination thereof. In some embodiments, R² is 1 O-acylated 3,5-dihydroxydecanoate or 3,5-dihydroxydodecanoate. In some embodiments, R² is 2 O-acylated 3,5-dihydroxydecanoate, 3,5-dihydroxydodecanoate, or a combination thereof. In some embodiments, R² is 3 O-acylated 3,5-dihydroxydecanoate, 3,5-dihydroxydodecanoate, or a combination thereof. In some embodiments, R² is 4 O-acylated 3,5-dihydroxydecanoate, 3,5-dihydroxydodecanoate, or a combination thereof. In some embodiments, R² is 5 O-acylated 3,5-dihydroxydecanoate, 3,5-dihydroxydodecanoate, or a combination thereof. In some embodiments, R² is 6 O-acylated 3,5-dihydroxydecanoate, 3,5-dihydroxydodecanoate, or a combination thereof. In some embodiments, R² is 7 O-acylated 3,5-dihydroxydecanoate, 3,5-dihydroxydodecanoate, or a combination thereof. In some embodiments, R² is 8 O-acylated 3,5-dihydroxydecanoate, 3,5-dihydroxydodecanoate, or a combination thereof. In some embodiments, R² is 9 O-acylated 3,5-dihydroxydecanoate, 3,5-dihydroxydodecanoate, or a combination thereof. In some embodiments, R² is 10 O-acylated 3,5-dihydroxydecanoate, 3,5-dihydroxydodecanoate, or a combination thereof.

In some embodiments, R² is

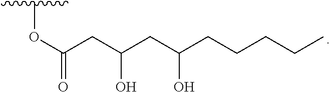 [SMILES: *OC(=O)CC(O)CC(O)CCCCC]

In some embodiments, R² is

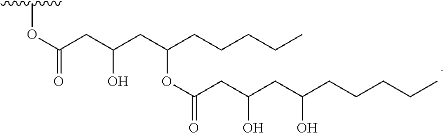 [SMILES: *OC(=O)CC(O)CC(CCCCC)OC(=O)CC(O)CC(O)CCCCC]

In some embodiments, R³ is L- or D-glycerol, L- or D-threitol, L- or D-erythritol, L- or D-arabitol, L- or D-xylitol, L- or D-lyxitol, L- or D-ribitol, L- or D-allitol, L- or D-altritol, L- or D-mannitol, L- or D-iditol, L- or D-gulitol, L- or D-glucitol, L- or D-galactitol, L- or D-talitol, 2-amino-D-mannitol, 2N-acetylamino-D-mannitol, L-rhamnitol, or D-fucitol. In some embodiments, R³ is L- or D-glycerol. In some embodiments, R³ is L-glycerol. In some embodiments, R³ is D-glycerol. In some embodiments, R³ is L- or D-threitol. In some embodiments, R³ is L-threitol. In some embodiments, R³ is D-threitol. In some embodiments, R³ is L- or D-erythritol. In some embodiments, R³ is L-erythritol. In some embodiments, R³ is D-erythritol. In some embodiments, R³ is L- or D-arabitol. In some embodiments, R³ is L-arabitol. In some embodiments, R³ is D-arabitol. In some embodiments, R³ is L- or D-xylitol. In some embodiments, R³ is L-xylitol. In some embodiments, R³ is D-xylitol. In some embodiments, R³ is L- or D-ribitol. In some embodiments, R³ is L-ribitol. In some embodiments, R³ is D-ribitol. In some embodiments, R³ is L- or D-allitol. In some embodiments, R³ is L-allitol. In some embodiments, R³ is D-allitol. In some embodiments, R³ is L- or D-altritol. In some embodiments, R³ is L-altritol. In some embodiments, R³ is D-altritol. In some embodiments, R³ is L- or D-mannitol. In some embodiments, R³ is L-mannitol. In some embodiments, R³ is D-mannitol. In some embodiments, R³ is L- or D-iditol. In some embodiments, R³ is L-iditol. In some embodiments, R³ is D-iditol. In some embodiments, R³ is L- or D-gulitol. In some embodiments, R³ is L-gulitol. In some embodiments, R³ is D-gulitol. In some embodiments, R³ is L- or D-glucitol. In some embodiments, R³ is L-glucitol. In some embodiments, R³ is D-glucitol. In some embodiments, R³ is L- or D-galactitol. In some embodiments, R³ is L-galactitol. In some embodiments, R³ is D-galactitol. In some embodiments, R³ is L- or D-talitol. In some embodiments, R³ is L-talitol. In some embodiments, R³ is D-talitol. In some embodiments, R³ is 2-amino-D-mannitol. In some embodiments, R³ is 2N-acetylamino-D-mannitol. In some embodiments, R³ is L-rhamnitol. In some embodiments, R³ is D-fucitol.

In specific embodiments, R³ is L- or D-mannitol, L- or D-glycerol, or L- or D-arabitol. In specific embodiments, R³ is L- or D-mannitol. In some embodiments, R³ is mannitol.

In some embodiments, R³ is H, such as when the one or more lipids do not comprise a carbohydrate/polyol headgroup.

In some embodiments, R⁴ is C₅-C₁₂ alkyl. In some embodiments, R⁴ is C₅-C₁₀ alkyl. In some embodiments, R⁴ is C₅-C₇ alkyl. In some embodiments, R⁴ is C₅ or C₇ alkyl. In some embodiments, R⁴ is C₅ alkyl and the one or more lipids represented by a structure of Formula (I) are represented by a structure of Formula (I-A). In some embodiments, R⁴ is C₇ alkyl and the one or more lipids represented by a structure of Formula (I) are represented by a structure of Formula (I-B).

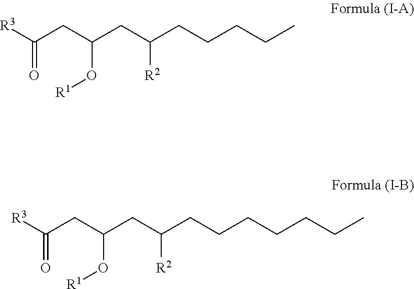

In some embodiments, the one or more lipids comprise a combination (e.g., mixture) of lipids. The one or more lipids may comprise one or more lipids described in Table 1.

TABLE 1

| Lipid | | Chemical |
|---|---|---|
| No. | Structure | Formula |
| 1 | 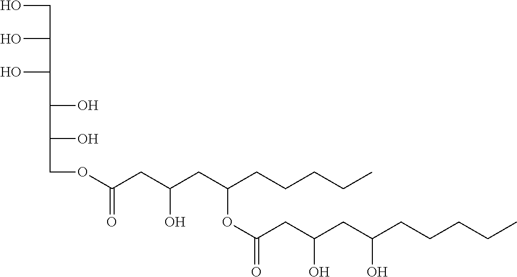 [SMILES: CCCCCC(O)CC(O)CC(=O)OC(CCCCC)CC(O)CC(=O)OCC(O)C(O)C(O)C(O)CO] | C₂₆H₅₀O₁₂ |
| 2 | 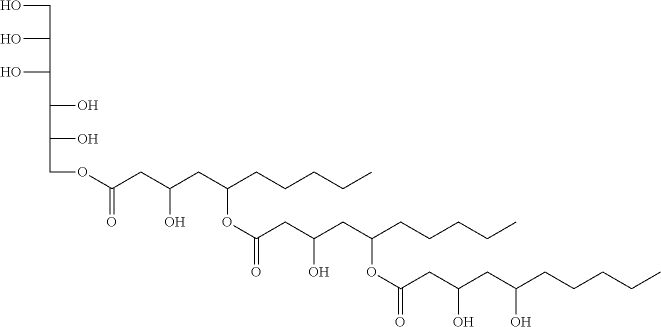 [SMILES: CCCCCC(O)CC(O)CC(=O)OC(CCCCC)CC(O)CC(=O)OC(CCCCC)CC(O)CC(=O)OCC(O)C(O)C(O)C(O)CO] | C₃₆H₆₈O₁₅ |
| 3 | 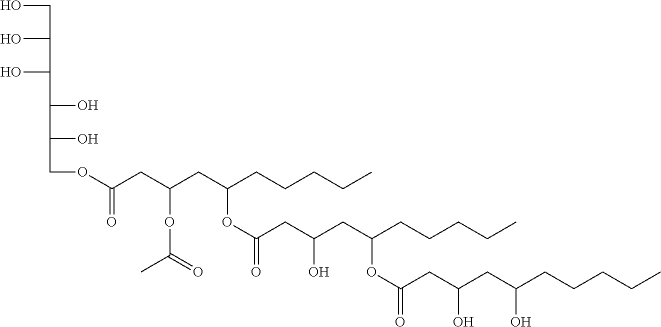 [SMILES: CCCCCC(O)CC(O)CC(=O)OC(CCCCC)CC(O)CC(=O)OC(CCCCC)CC(CC(=O)OCC(O)C(O)C(O)C(O)CO)OC(C)=O] | C₃₈H₇₀O₁₆ |
| 4 | 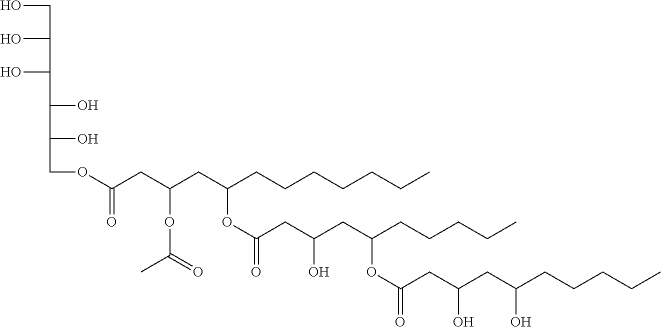 [SMILES: CCCCCCCC(CC(CC(=O)OCC(O)C(O)C(O)C(O)CO)OC(C)=O)OC(=O)CC(O)CC(CCCCC)OC(=O)CC(O)CC(O)CCCCC] | C₄₀H₇₄O₁₆ |
| 5 | 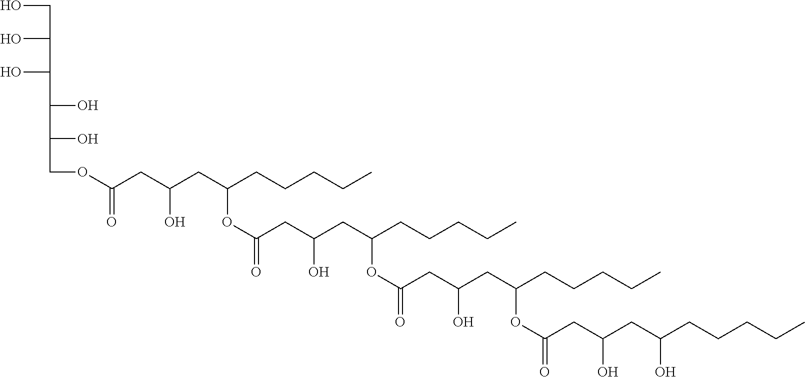 [SMILES: CCCCCC(O)CC(O)CC(=O)OC(CCCCC)CC(O)CC(=O)OC(CCCCC)CC(O)CC(=O)OC(CCCCC)CC(O)CC(=O)OCC(O)C(O)C(O)C(O)CO] | C₄₆H₈₆O₁₈ |

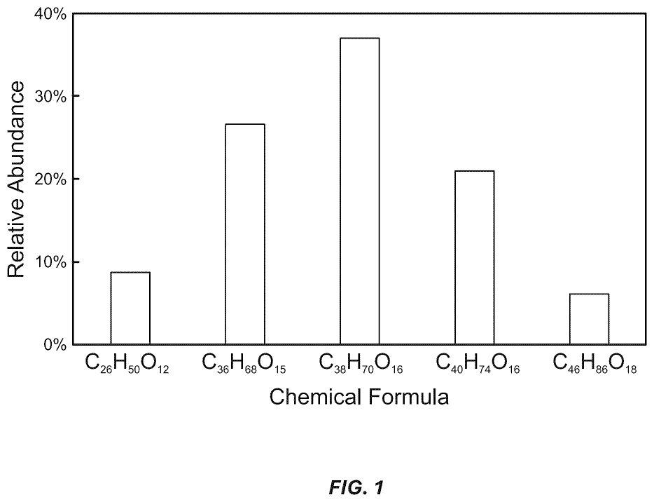

In some embodiments, the one or more lipids comprise lipid 1 (as described in Table 1) in an amount of at least 5 wt % (e.g., relative to the total amount of the one more lipids). In some embodiments, the one or more lipids comprise lipid 1 (as described in Table 1) in an amount of at least about 2 wt %, about 4 wt %, about 5 wt %, about 6 wt %, about 7 wt %, about 8 wt %, about 9 wt %, about 10 wt %, about 12 wt %, or about 14 wt %. In some embodiments, the one or more lipids comprise lipid 1 (as described in Table 1) in an amount of at most about 14 wt %, about 12 wt %, about 10 wt %, about 9 wt %, about 8 wt %, about 7 wt %, about 6 wt %, about 5 wt %, about 4 wt %, or about 2 wt %. In some embodiments, the one or more lipids comprise lipid 1 (as described in Table 1) in an amount of about 2 wt % to about 14 wt %, about 4 wt % to about 12 wt %, about 7 wt % to about 12 wt %, about 8 wt % to about 10 wt %, about 5 wt % to about 9 wt %, or about 7 wt % to about 9 wt %. In some embodiments, the one or more lipids comprise lipid 1 (as described in Table 1) in an amount of about 2 wt %, about 4 wt %, about 5 wt %, about 6 wt %, about 7 wt %, about 8 wt %, about 9 wt %, about 10 wt %, about 12 wt %, or about 14 wt %. In some embodiments, the one or more lipids comprise lipid 1 (as described in Table 1) in an amount of about 9 wt %, such as described in FIG. 1.

In some embodiments, the one or more lipids comprise lipid 2 (as described in Table 1) in an amount of at least 20 wt % (e.g., relative to the total amount of the one or more lipids). In some embodiments, the one or more lipids comprise lipid 2 (as described in Table 1) in an amount of at least about 15 wt %, about 17 wt %, about 20 wt %, about 22 wt %, about 24 wt %, about 26 wt %, about 28 wt %, about 30 wt %, about 32 wt %, or about 34 wt %. In some embodiments, the one or more lipids comprise lipid 2 (as described in Table 1) in an amount of at most about 34 wt %, about 32 wt %, about 30 wt %, about 28 wt %, about 26 wt %, about 24 wt %, about 22 wt %, about 20 wt %, about 17 wt %, or about 15 wt %. In some embodiments, the one or more lipids comprise lipid 2 (as described in Table 1) in an amount of about 15 wt % to about 34 wt %, about 17 wt % to about 32 wt %, about 22 wt % to about 30 wt %, about 24 wt % to about 28 wt %, about 17 wt % to about 26 wt %, or about 22 wt % to about 26 wt %. In some embodiments, the one or more lipids comprise lipid 2 (as described in Table 1) in an amount of about 15 wt %, about 17 wt %, about 20 wt %, about 22 wt %, about 24 wt %, about 26 wt %, about 28 wt %, about 30 wt %, about 32 wt %, or about 34 wt %. In some embodiments, the one or more lipids comprise lipid 2 (as described in Table 1) in an amount of about 27 wt %, such as described in FIG. 1.

In some embodiments, the one or more lipids comprise lipid 3 (as described in Table 1) in an amount of at least 30 wt % (e.g., relative to the total amount of the one or more lipids). In some embodiments, the one or more lipids comprise lipid 3 (as described in Table 1) in an amount of at least about 25 wt %, about 27 wt %, about 30 wt %, about 32 wt %, about 34 wt %, about 36 wt %, about 38 wt %, about 40 wt %, about 42 wt %, or about 44 wt %. In some embodiments, the one or more lipids comprise lipid 3 (as described in Table 1) in an amount of at most about 44 wt %, about 42 wt %, about 40 wt %, about 38 wt %, about 36 wt %, about 34 wt %, about 32 wt %, about 30 wt %, about 27 wt %, or about 25 wt %. In some embodiments, the one or more lipids comprise lipid 3 (as described in Table 1) in an amount of about 25 wt % to about 44 wt %, about 27 wt % to about 42 wt %, about 32 wt % to about 40 wt %, about 34 wt % to about 38 wt %, about 27 wt % to about 36 wt %, or about 32 wt % to about 36 wt %. In some embodiments, the one or more lipids comprise lipid 3 (as described in Table 1) in an amount of about 25 wt %, about 27 wt %, about 30 wt %, about 32 wt %, about 34 wt %, about 36 wt %, about 38 wt %, about 40 wt %, about 42 wt %, or about 44 wt %. In some embodiments, the one or more lipids comprise lipid 3 (as described in Table 1) in an amount of about 37 wt %, such as described in FIG. 1.

In some embodiments, the one or more lipids comprise lipid 4 (as described in Table 1) in an amount of at least 15 wt % (e.g., relative to the total amount of the one or more lipids). In some embodiments, the one or more lipids comprise lipid 4 (as described in Table 1) in an amount of at least about 10 wt %, about 12 wt %, about 14 wt %, about 16 wt %, about 18 wt %, about 20 wt %, about 22 wt %, about 24 wt %, about 26 wt %, or about 28 wt %. In some embodiments, the one or more lipids comprise lipid 4 (as described in Table 1) in an amount of at most about 28 wt %, about 26 wt %, about 24 wt %, about 22 wt %, about 20 wt %, about 18 wt %, about 16 wt %, about 14 wt %, about 12 wt %, or about 10 wt %. In some embodiments, the one or more lipids comprise lipid 4 (as described in Table 1) in an amount of about 10 wt % to about 28 wt %, about 12 wt % to about 26 wt %, about 16 wt % to about 24 wt %, about 18 wt % to about 22 wt %, about 12 wt % to about 20 wt %, or about 16 wt % to about 20 wt %. In some embodiments, the one or more lipids comprise lipid 4 (as described in Table 1) in an amount of about 10 wt %, about 12 wt %, about 14 wt %, about 16 wt %, about 18 wt %, about 20 wt %, about 22 wt %, about 24 wt %, about 26 wt %, or about 28 wt %. In some embodiments, the one or more lipids comprise lipid 4 (as described in Table 1) in an amount of about 21 wt %, such as shown in FIG. 1.

In some embodiments, the one or more lipids comprise lipid 5 (as described in Table 1) in an amount of at least 3 wt % (e.g., relative to the total amount of the one or more lipids). In some embodiments, the one or more lipids comprise lipid 5 (as described in Table 1) in an amount of at least about 1 wt %, about 2 wt %, about 3 wt %, about 4 wt %, about 5 wt %, about 6 wt %, about 7 wt %, about 8 wt %, about 9 wt %, or about 10 wt %. In some embodiments, the one or more lipids comprise lipid 5 (as described in Table 1) in an amount of at most about 10 wt %, about 9 wt %, about 8 wt %, about 7 wt %, about 6 wt %, about 5 wt %, about 4 wt %, about 3 wt %, about 2 wt %, or about 1 wt %. In some embodiments, the one or more lipids comprise lipid 5 (as described in Table 1) in an amount of about 1 wt % to about 10 wt %, about 2 wt % to about 9 wt %, about 4 wt % to about 8 wt %, about 5 wt % to about 7 wt %, about 2 wt % to about 6 wt %, or about 4 wt % to about 6 wt %. In some embodiments, the one or more lipids comprise lipid 5 (as described in Table 1) in an amount of about 1 wt %, about 2 wt %, about 3 wt %, about 4 wt %, about 5 wt %, about 6 wt %, about 7 wt %, about 8 wt %, about 9 wt %, or about 10 wt %. In some embodiments, the one or more lipids comprise lipid 5 (as described in Table 1) in an amount of about 6 wt %, such as shown in FIG. 1.

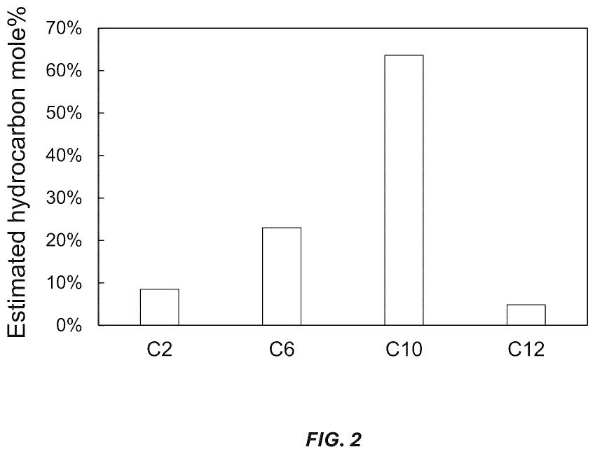

In some embodiments, the ratio of C₁₂:C₁₀ alkyl chains in the lipid bioproduct or compositions may be from about 1:8 to about 1:15. In some embodiments, the ratio of C₁₂:C₁₀ alkyl chains may be about 1:8, 1:9, 1:10, 1:11, 1:12, or 1:13. In some embodiments, the ratio of C₁₂:C₁₀ alkyl chains may be about 1:13, such as described in FIG. 2.

In some embodiments, the relative amounts of lipids described in Table 1 may be determined by mass spectrometry, such as TOF-MS.

In some embodiments, the one or more lipids as described herein is prepared by an organism. In some embodiments, the organism is a microorganism.

In some embodiments, the one or more lipids as described herein is bio-derived. In some embodiments, the one or more lipids is produced by an organism.

In some embodiments, the organism is within the family Dothioraceae. In some embodiments, the organism is within the genus Aureobasidium. In some embodiments, the organism is from the species aleuritis, apocryptum, dalgeri, harposporum, indicum, iranianum, khasianum, leucospermi, lilii, mangrovei, melanogenum, microstictum, namibiae, nigrum, pini, proteae, prunicola, pullulans, ribis, sanguinariae, subglaciale, thailandense, thujae-plicatae, tremulum, umbellulariae, or vaccinii. In some embodiments, the organism is from the species pullulans, melanogenum, or thailandense. In some embodiments, the organism is Aureobasidium pullulans. In some embodiments the organism is genetically modified. In some embodiments, the organism is Aureobasidium pullulans or a genetically modified version thereof. In some embodiments, a genetically modified version of the organism comprises a modification to a gene related to polymalic acid (e.g., PMA synthase), pullulan production (e.g., UDPG-pyrophosphorylase), oxygen binding hemoglobin (e.g., Vhb), a pathway activator gene (e.g., pacC), or a combination thereof. In some embodiments, a genetically modified version of the organism comprises knockout of polymalic acid (e.g., PMA synthase), knockout of pullulan production (e.g., UDPG-pyrophosphorylase), expression of oxygen binding hemoglobin (e.g., Vhb), overexpression of pathway activator gene (e.g., pacC), or a combination thereof. In some embodiments, the organism produces at least 50 g/L titer of the one or more lipids.

In some embodiments, the bioproduct is pre-treated to remove cellular debris, water, or other trace components prior to the distilling or contacting (e.g., saponifying or acidifying) steps. In some embodiments, the methods comprise collecting the one or more lipids, such as the one or more lipids described elsewhere herein. In some embodiments, the methods comprise collecting the one or more lipids from the bioproduct. In some embodiments, the collecting comprises liquid-liquid extraction, distillation, physical separation, mechanical cell disruption, supercritical fluid extraction, gravimetric separation, flash evaporation, or a combination thereof. In some embodiments, the collecting comprises liquid-liquid extraction. In some embodiments, the collecting comprises distillation. In some embodiments, the collecting comprises physical separation. In some embodiments, the collecting comprises mechanical cell disruption. In some embodiments, the collecting comprises supercritical fluid extraction. In some embodiments, the collecting comprises gravimetric separation. In some embodiments, the collecting comprises flash evaporation. In some embodiments, the collecting comprises liquid-liquid extraction, distillation, physical separation, and flash evaporation.

In some embodiments, the liquid-liquid extraction comprises use of a solvent. The solvent may be an organic solvent. The solvent may be a mixture of organic solvents. In some embodiments, the solvent is chloroform, methanol, ethanol, propanol, butanol, isopropanol, hexane, toluene, petroleum ether, methyl ethyl ketone (MEK), acetonitrile, ethyl acetate, or a combination thereof. In some embodiments, the solvent is chloroform. In some embodiments, the solvent is methanol. In some embodiments, the solvent is ethanol. In some embodiments, the solvent is proanol. In some embodiments, the solvent is butanol. In some embodiments, the solvent is isopropanol. In some embodiments, the solvent is hexane. In some embodiments, the solvent is toluene. In some embodiments, the solvent is petroleum ether. In some embodiments, the solvent is methyl ethyl ketone (MEK). In some embodiments, the solvent is acetonitrile. In some embodiments, the solvent is ethyl acetate. In some embodiments, the solvent is removed during the distilling.

In some embodiments, waste streams produced from the extraction include solvent (e.g., organic solvent), organic/cellular residue, or a combination thereof.

In some embodiments, the liquid-liquid extraction comprises countercurrent liquid extraction, batch-wise (e.g., single stage) extraction, ultrasound-assisted extraction, or multistage countercurrent continuous extraction. In some embodiments, the liquid-liquid extraction comprises countercurrent liquid extraction.

In some embodiments, the liquid-liquid extraction is completed at any suitable temperature. In some embodiments, the liquid-liquid extraction is completed at a temperature of from about 25° C. to about 60° C. In some embodiments, the liquid-liquid extraction is completed at a temperature of from about 35° C. to about 50° C. In some embodiments, the liquid-liquid extraction is completed at a temperature of at least about 20° C., about 25° C., about 30° C., about 35° C., about 40° C., about 45° C., about 50° C., about 55° C., about 60° C., or about 65° C. In some embodiments, the liquid-liquid extraction is completed at a temperature of at most about 65° C., about 60° C., about 55° C., about 50° C., about 45° C., about 40° C., about 35° C., about 30° C., about 25° C., or about 20° C. In some embodiments, the liquid-liquid extraction is completed at a temperature of from about 20° C. to about 65° C., about 25° C. to about 65° C., about 25° C. to about 60° C., about 30° C. to about 60° C., about 20° C. to about 50° C., about 30° C. to about 50° C., or about 35° C. to about 55° C. In some embodiments, the liquid-liquid extraction is completed at a temperature of about 20° C., about 25° C., about 30° C., about 35° C., about 40° C., about 45° C., about 50° C., about 55° C., about 60° C., or about 65° C.

In some embodiments, the counter current liquid extraction comprises counter current liquid-liquid extraction. In some embodiments, the countercurrent liquid-liquid extraction is continuous countercurrent liquid-liquid extraction. In some embodiments, the liquid-liquid extraction is completed with a dynamic flow reactor.

In some embodiments, the distillation is water stripping distillation, steam distillation, vacuum distillation, azeotropic distillation, fractional distillation, or simple distillation. In some embodiments, the distillation is water stripping distillation. In some embodiments, the distillation is steam distillation. In some embodiments, the distillation is vacuum distillation. In some embodiments, the vacuum distillation is wiped-film distillation. In some embodiments, the distillation is azeotropic distillation. In some embodiments, the distillation is fractional distillation. In some embodiments, the distillation is simple distillation.

In some embodiments, the distillation comprises use of a reboiler and a condenser.

In some embodiments, the reboiler comprises a temperature of from about 80° C. to about 120° C. In some embodiments, the reboiler comprises a temperature of from about 90° C. to about 110° C. In some embodiments, the reboiler comprises a temperature of at least about 75° C., about 80° C., about 85° C., about 90° C., about 95° C., about 100° C., about 105° C., about 110° C., about 115° C., or about 120° C. In some embodiments, the reboiler comprises a temperature of at most about 120° C., about 115° C., about 110° C., about 105° C., about 100° C., about 95° C., about 90° C., about 85° C., about 80° C., or about 75° C. In some embodiments, the reboiler comprises a temperature of from about 75° C. to about 120° C., about 80° C. to about 120° C., about 80° C. to about 115° C., about 85° C. to about 115° C., about 75° C. to about 105° C., about 85° C. to about 105° C., or about 90° C. to about 110° C. In some embodiments, the reboiler comprises a temperature of about 75° C., about 80° C., about 85° C., about 90° C., about 95° C., about 100° C., about 105° C., about 110° C., about 115° C., or about 120° C.

In some embodiments, the condenser comprises a temperature of from about 50° C. to about 90° C. In some embodiments, the condenser comprises a temperature of from about 60° C. to about 80° C. In some embodiments, the condenser comprises a temperature of at least about 50° C., about 55° C., about 60° C., about 65° C., about 70° C., about 75° C., about 80° C., about 85° C., about 90° C., or about 95° C. In some embodiments, the condenser comprises a temperature of at most about 95° C., about 90° C., about 85° C., about 80° C., about 75° C., about 70° C., about 65° C., about 60° C., about 55° C., or about 50° C. In some embodiments, the condenser comprises a temperature of from about 50° C. to about 95° C., about 55° C. to about 95° C., about 55° C. to about 90° C., about 60° C. to about 90° C., about 50° C. to about 80° C., about 60° C. to about 80° C., or about 65° C. to about 85° C. In some embodiments, the condenser comprises a temperature of about 50° C., about 55° C., about 60° C., about 65° C., about 70° C., about 75° C., about 80° C., about 85° C., about 90° C., or about 95° C.

In some embodiments, the physical separation comprises centrifugation, decantation, or a combination thereof. In some embodiments, the physical separation comprises centrifugation. In some embodiments, the physical separation comprises decantation.

In some embodiments, the physical separation is completed in ambient conditions. In some embodiments, the physical separation is completed at a temperature of from about 25° C. to about 60° C. In some embodiments, the physical separation is completed at a temperature of from about 35° C. to about 50° C. In some embodiments, the physical separation is completed at a temperature of at least about 25° C., about 30° C., about 35° C., about 40° C., about 45° C., about 50° C., about 55° C., about 60° C., about 65° C., or about 70° C. In some embodiments, the physical separation is completed at a temperature of at most about 70° C., about 65° C., about 60° C., about 55° C., about 50° C., about 45° C., about 40° C., about 35° C., about 30° C., or about 25° C. In some embodiments, the physical separation is completed at a temperature of from about 25° C. to about 70° C., about 30° C. to about 70° C., about 30° C. to about 65° C., about 35° C. to about 65° C., about 25° C. to about 55° C., about 35° C. to about 55° C., or about 40° C. to about 60° C. In some embodiments, the physical separation is completed at a temperature of about 25° C., about 30° C., about 35° C., about 40° C., about 45° C., about 50° C., about 55° C., about 60° C., about 65° C., or about 70° C.

In some embodiments, the centrifugation is completed in ambient conditions. In some embodiments, the centrifugation is completed at a temperature of from about 0° C. to about 60° C. In some embodiments, the centrifugation is completed at a temperature of from about 35° C. to about 50° C. In some embodiments, the centrifugation is completed at a temperature of at least about 0° C., 5° C., 10° C., 15° C., 20° C., 25° C., about 30° C., about 35° C., about 40° C., about 45° C., about 50° C., about 55° C., about 60° C., about 65° C., or about 70° C. In some embodiments, the centrifugation is completed at a temperature of at most about 70° C., about 65° C., about 60° C., about 55° C., about 50° C., about 45° C., about 40° C., about 35° C., about 30° C., or about 25° C. In some embodiments, the centrifugation is completed at a temperature of from about 25° C. to about 70° C., about 30° C. to about 70° C., about 30° C. to about 65° C., about 35° C. to about 65° C., about 25° C. to about 55° C., about 35° C. to about 55° C., or about 40° C. to about 60° C. In some embodiments, the centrifugation is completed at a temperature of about 25° C., about 30° C., about 35° C., about 40° C., about 45° C., about 50° C., about 55° C., about 60° C., about 65° C., or about 70° C.

In some embodiments, the centrifugation comprises disc stack centrifugation. In some embodiments, the centrifugation comprises use of a three phase decanter centrifuge.

In some embodiments, the collecting comprises flash evaporation. In some embodiments, the flash evaporation comprises use of a flash drum. In some embodiments, the flash evaporation is completed at any suitable temperature. In some embodiments, the flash evaporation is completed at a temperature of from about 35° C. to about 55° C. In some embodiments, the flash evaporation is completed at a temperature of at least about 35° C., about 40° C., about 45° C., about 50° C., about 55° C., about 60° C., about 65° C., about 70° C., about 75° C., or about 80° C. In some embodiments, the flash evaporation is completed at a temperature of at most about 80° C., about 75° C., about 70° C., about 65° C., about 60° C., about 55° C., about 50° C., about 45° C., about 40° C., or about 35° C. In some embodiments, the flash evaporation is completed at a temperature of from about 35° C. to about 80° C., about 40° C. to about 80° C., about 40° C. to about 75° C., about 45° C. to about 75° C., about 35° C. to about 65° C., about 45° C. to about 65° C., or about 50° C. to about 70° C. In some embodiments, the flash evaporation is completed at a temperature of about 35° C., about 40° C., about 45° C., about 50° C., about 55° C., about 60° C., about 65° C., about 70° C., about 75° C., or about 80° C.

In some embodiments, before distillation, the method comprises solvent-extraction, such as to purify the one or more lipids provided herein. The solvent extraction may remove or reduce cellular debris, water, or other trace components. The solvent extraction may introduce organic solvent which may be removed and/or recovered during the subsequent distillation.

### Distillation and Pretreatment

In some embodiments, the method comprises distillation. In some embodiments, the method comprises distilling. In some embodiments, the method comprises reacting the one or more lipids and distilling. In some embodiments, the method comprises reacting and distilling after the collecting of the one or more lipids (e.g., after preparation by the organism). In some embodiments, the reacting the bioproduct and distilling produces the one or more substituted or unsubstituted (e.g., saturated or unsaturated) lactones. In some embodiments, the method comprises reacting the lipids produced by the organism described elsewhere herein. In some embodiments, the distilling provides one or more substituted or unsubstituted (e.g., saturated or unsaturated) lactones. In some embodiments, the method comprises reacting at least a portion of the one or more lipids to provide one or more substituted or unsubstituted (e.g., saturated or unsaturated) lactones. In some embodiments, the method comprises reacting at least a portion of the one or more lipids and distilling to provide the 5-hydroxy-2-decenoic acid-δ-lactone and 3,5-dihydroxydecanoic acid-δ-lactone.

In some embodiments, the method comprises saponification. In some embodiments, the method comprises saponifying. In some embodiments, the saponifying comprises contacting at least a portion of the lipids with a base (e.g., prior to distilling). In some embodiments, the base is potassium hydroxide, sodium hydroxide, calcium hydroxide, lithium hydroxide, magnesium hydroxide, strontium hydroxide, barium hydroxide, potassium oxide, sodium oxide, calcium oxide, lithium oxide, magnesium oxide, strontium oxide, barium oxide, a basic clay mineral (e.g., hydrotalcite), or a combination thereof. In some embodiments, the base is potassium hydroxide. In some embodiments, the base is sodium hydroxide. In some embodiments, the base is calcium hydroxide.

In some embodiments, the contacting with the base is before the distilling. In some embodiments, the contacting with the base occurs concurrently with the distilling. In some embodiments, the base is removed from the resulting composition before distilling. In some embodiments, the base is not removed from the resulting composition before distilling.

In some embodiments, the saponifying is completed at a temperature of at least 40° C. In some embodiments, the saponifying is completed at a temperature of about 30° C. to about 50° C. In some embodiments, the saponifying is completed at a temperature of about 25° C. to about 55° C., about 25° C. to about 60° C., about 30° C. to about 65° C., about 30° C. to about 70° C., about 40° C. to about 65° C., about 40° C. to about 70° C., about 40° C. to about 80° C., about 40° C. to about 90° C., about 40° C. to about 100° C. In some embodiments, the saponifying is completed at a temperature of about 25° C., about 30° C., about 40° C., about 45° C., about 50° C., about 55° C., about 60° C., about 65° C., about 70° C., about 75° C., about 80° C., or about 90° C. In some embodiments, the saponifying is completed at a temperature of from about 25° C. to about 90° C. In some embodiments, the saponifying is completed at a temperature of at least about 25° C., about 30° C., about 40° C., about 45° C., about 50° C., about 55° C., about 60° C., about 65° C., about 70° C., about 75° C., about 80° C., or about 90° C. In some embodiments, the saponifying is completed at a temperature of at least 40° C. In some embodiments, the saponifying is completed at a temperature of at most about 60° C., about 65° C., about 70° C., about 75° C., about 80° C., or about 90° C. In some embodiments, the saponifying is completed at a temperature of less than 40° C.

In some embodiments, the method comprises contacting at least a portion of the one or more lipids with a catalyst (e.g., prior to distilling). In some embodiments, the method comprises contacting at least a portion of the one or more lipids with an acid catalyst. In some embodiments, the contacting with a catalyst results in lactone formation.

In some embodiments, the acid catalyst is a weak acid (e.g., with a pKₐ of between about 4 and about 7). In some embodiments, the acid catalyst is a strong acid (e.g., with a pKₐ of less than about 4). In some embodiments, the acid catalyst is a mineral acid. In some embodiments, the acid catalyst is an organic acid. In some embodiments, the acid catalyst is formic acid, acetic acid, propionic acid, oxalic acid, maleic acid, succinic acid, malonic acid, or benzoic acid. In some embodiments, the acid catalyst has a pKₐ of less than about 5. In some embodiments, the acid catalyst is hydrochloric acid, sulfuric acid, hydrobromic acid, phosphoric acid, iodic acid, nitric acid, perchloric acid, or a combination thereof. In some embodiments, the acid catalyst is hydrochloric acid. In some embodiments, the acid catalyst is sulfuric acid. In some embodiments, the acid catalyst is hydrobromic acid. In some embodiments, the acid catalyst is phosphoric acid. In some embodiments, the acid catalyst is iodic acid. In some embodiments, the acid catalyst may be a solid acid, such as a zeolite, polyoxometallate, treated or untreated metal oxides such as titania or alumina, niobic acid, or ion exchange resin.

In some embodiments, waste streams produced from the reacting and/or distilling include salts resulting from the acid/base reactions as described herein (e.g., from the saponification or lactonization).

In some embodiments, the method does not comprise contacting at least a portion of the one or more lipids with a catalyst (e.g., an acid catalyst, such as sulfuric acid). In some embodiments, the method comprises saponifying (e.g., with a base) but does not comprise acidifying. In some embodiments, the method comprises saponifying (e.g., with a base) and thermally forming the resulting lactones.

In some embodiments, the contacting with the acid catalyst is before the distilling. In some embodiments, the contacting with the acid catalyst occurs concurrently with the distilling. In some embodiments, the acid catalyst is removed from the resulting composition before distilling. In some embodiments, the acid catalyst is not removed from the resulting composition before distilling.

In some embodiments, the method comprises of contacting at least a portion of the lipid mixture with a weak base following the contacting with an acid catalyst. In some embodiments the weak base is potassium bicarbonate, sodium bicarbonate, sodium carbonate, calcium carbonate, sodium acetate, sodium citrate, sodium borate, magnesium hydroxide, or ammonia. In some embodiments, the weak base is potassium bicarbonate. In some embodiments, the weak base is sodium bicarbonate. In some embodiments, the weak base is removed from the resulting composition before distilling. In some embodiments, the weak base is not removed from the resulting composition before distilling.

In some embodiments, the distilling comprises simple distillation, fractional distillation, vacuum distillation, azeotropic distillation, spinning band distillation, wiped film distillation, or a combination thereof. In some embodiments, the distilling comprises simple distillation. In some embodiments, the distilling comprises fractional distillation. In some embodiments, the distilling comprises vacuum distillation. In some embodiments, the distilling comprises spinning band distillation. In some embodiments, the distilling comprises azeotropic distillation. In some embodiments, the distilling comprises wiped film distillation.

In some embodiments, the methods provided herein comprise wiped film distillation of at least a portion of the one or more lipids to provide one or more substituted or unsubstituted (e.g., saturated or unsaturated) lactones. In some embodiments, the methods provided herein comprise simple distillation of at least a portion of the one or more lipids to provide one or more substituted or unsubstituted (e.g., saturated or unsaturated) lactones. In some embodiments, the methods provided herein comprise fractional distillation of at least a portion of the one or more lipids to provide one or more substituted or unsubstituted (e.g., saturated or unsaturated) lactones.

In some embodiments, the distilling is completed at a temperature of at least 200° C. In some embodiments, the distilling is completed at a temperature of about 180° C. to about 290° C. In some embodiments, the distilling is completed at a temperature of about 180° C. to about 230° C., about 180° C. to about 290° C., about 190° C. to about 230° C., about 190° C. to about 240° C., about 200° C. to about 230° C., about 200° C. to about 240° C., about 200° C. to about 250° C., about 200° C. to about 270° C., about 200° C. to about 290° C., about 210° C. to about 230° C., about 210° C. to about 240° C., about 210° C. to about 250° C., about 210° C. to about 270° C., about 210° C. to about 290° C., about 215° C. to about 230° C., about 215° C. to about 240° C., about 215° C. to about 250° C., about 215° C. to about 270° C., about 215° C. to about 290° C., about 220° C. to about 230° C., about 220° C. to about 240° C., about 220° C. to about 250° C., about 225° C. to about 230° C., about 225° C. to about 240° C., about 225° C. to about 250° C., about 230° C. to about 240° C., or about 230° C. to about 250° C. In some embodiments, the distilling is completed at a temperature of about 180° C., about 190° C., about 200° C., about 210° C., about 215° C., about 220° C., about 225° C., about 230° C., about 240° C., about 250° C., about 270° C., or about 290° C. In some embodiments, the distilling is completed at a temperature of from about 200° C. to about 250° C. In some embodiments, the distilling is completed at a temperature of from about 215° C. to about 235° C. In some embodiments, the distilling is completed at a temperature of at least about 180° C., about 190° C., about 200° C., about 210° C., about 215° C., about 220° C., about 225° C., about 230° C., about 240° C., about 250° C., or about 270° C. In some embodiments, the distilling is completed at a temperature of at least 200° C. In some embodiments, the distilling is completed at a temperature of at most about 225° C., about 230° C., about 240° C., about 250° C., about 270° C., or about 290° C. In some embodiments, the distilling is completed at a temperature of at least 250° C. (e.g., to thermally form the lactones). In some embodiments, the distilling is completed at a temperature of less than 200° C.

In some embodiments, the wiped film distillation is completed at a temperature of at least 200° C. In some embodiments, the wiped film distillation is completed at a temperature of about 180° C. to about 290° C. In some embodiments, the wiped film distillation is completed at a temperature of about 180° C. to about 230° C., about 180° C. to about 290° C., about 190° C. to about 230° C., about 190° C. to about 240° C., about 200° C. to about 230° C., about 200° C. to about 240° C., about 200° C. to about 250° C., about 200° C. to about 270° C., about 200° C. to about 290° C., about 210° C. to about 230° C., about 210° C. to about 240° C., about 210° C. to about 250° C., about 210° C. to about 270° C., about 210° C. to about 290° C., about 215° C. to about 230° C., about 215° C. to about 240° C., about 215° C. to about 250° C., about 215° C. to about 270° C., about 215° C. to about 290° C., about 220° C. to about 230° C., about 220° C. to about 240° C., about 220° C. to about 250° C., about 225° C. to about 230° C., about 225° C. to about 240° C., about 225° C. to about 250° C., about 230° C. to about 240° C., or about 230° C. to about 250° C. In some embodiments, the wiped film distillation is completed at a temperature of about 180° C., about 190° C., about 200° C., about 210° C., about 215° C., about 220° C., about 225° C., about 230° C., about 240° C., about 250° C., about 270° C., or about 290° C. In some embodiments, the wiped film distillation is completed at a temperature of from about 200° C. to about 250° C. In some embodiments, the wiped film distillation is completed at a temperature of from about 215° C. to about 235° C. In some embodiments, the wiped film distillation is completed at a temperature of at least about 180° C., about 190° C., about 200° C., about 210° C., about 215° C., about 220° C., about 225° C., about 230° C., about 240° C., about 250° C., or about 270° C. In some embodiments, the wiped film distillation is completed at a temperature of at least 200° C. In some embodiments, the wiped film distillation is completed at a temperature of at most about 225° C., about 230° C., about 240° C., about 250° C., about 270° C., or about 290° C. In some embodiments, the wiped film distillation is completed at a temperature of about 225° C.

In some embodiments, the distilling is completed at a temperature of less than about 200° C. In some embodiments, the distilling is completed at a temperature of at least about 100° C., about 110° C., about 120° C., about 130° C., about 140° C., about 150° C., about 160° C., about 180° C., about 190° C., or about 200° C. In some embodiments, the distilling is completed at a temperature of at most about 200° C., about 190° C., about 180° C., about 160° C., about 150° C., about 140° C., about 130° C., about 120° C., about 110° C., or about 100° C. In some embodiments, the distilling is completed at a temperature of from about 100° C. to about 200° C., about 110° C. to about 200° C., about 110° C. to about 190° C., about 120° C. to about 190° C., about 100° C. to about 160° C., about 120° C. to about 160° C., or about 130° C. to about 180° C. In some embodiments, the distilling is completed at a temperature of about 100° C., about 110° C., about 120° C., about 130° C., about 140° C., about 150° C., about 160° C., about 180° C., about 190° C., or about 200° C.

In some embodiments, the distillation is completed at reduced pressure. In some embodiments, the distillation is completed at a pressure of less than 1 atm. In some embodiments, the distillation is completed at a pressure of less than 0.5 atm. In some embodiments, the distillation is completed at a pressure of less than 0.1 atm. In some embodiments, the distillation is completed in a vacuum. In some embodiments, the distillation is completed at about 1 atm.

In some embodiments, the reacting and/or distilling provides a yield of the one or more substituted or unsubstituted (e.g., saturated or unsaturated) lactones of at least 30 wt %. In some embodiments, the reacting and/or distilling provides a yield of the one or more substituted or unsubstituted (e.g., saturated or unsaturated) lactones of at least 50 wt %. In some embodiments, the reacting and/or distilling provides a yield of the one or more substituted or unsubstituted (e.g., saturated or unsaturated) lactones of at least 70 wt %. In some embodiments, the reacting and/or distilling provides a yield of the one or more substituted or unsubstituted (e.g., saturated or unsaturated) lactones of about 30 wt %, 35 wt %, 40 wt %, 45 wt %, 50 wt %, 55 wt %, 60 wt %, 65 wt %, 70 wt %, 75 wt %, 80 wt %, 85 wt %, 90 wt %, or 95 wt %.

In some embodiments, the reacting and/or distilling provides mannitol (e.g., carbohydrate), a derivative of mannitol (e.g. C₅₋₆ molecules formed from the reaction of the carbohydrate with base or acid catalysts mentioned herein), and the one or more lactones, or a combination thereof.

In some embodiments, waste streams produced from the reacting and/or distilling include water vapor.

The one or more lactones (e.g., 5-hydroxy-2-decenoic acid-δ-lactone and 3,5-dihydroxydecanoic acid-δ-lactone) produced by the methods herein may have a CI of less than 1 kg CO₂e/kg of lactone. The one or more lactones (e.g., 5-hydroxy-2-decenoic acid-δ-lactone and 3,5-dihydroxydecanoic acid-δ-lactone) produced by the methods herein may have a CI of less than 0.9 kg CO₂e/kg of lactone, 0.8 kg CO₂e/kg of lactone, 0.7 kg CO₂e/kg of lactone, 0.7, kg CO₂e/kg of lactone, 0.5 kg CO₂e/kg of lactone, 0.4 kg CO₂e/kg of lactone, 0.3 kg CO₂e/kg of lactone, or 0.2 kg CO₂e/kg of lactone. The one or more lactones (e.g., 5-hydroxy-2-decenoic acid-δ-lactone and 3,5-dihydroxydecanoic acid-δ-lactone) produced by the methods herein may have a CI of less than 0.1 kg CO₂e/kg of lactone. In some cases, the one or more lactones (e.g., 5-hydroxy-2-decenoic acid-δ-lactone and 3,5-dihydroxydecanoic acid-δ-lactone) produced by the methods herein may have a CI of less than 0 kg CO₂e/kg of lactone, e.g., a negative CI.

In some embodiments, the method comprises a continuous production process. In some embodiments, the method comprises a continuous production process comprising one or more of the reacting, distilling, contacting, saponifying, or acidifying. In some embodiments, the continuous production process comprises the reacting. In some embodiments, the continuous production process comprises the distilling. In some embodiments, the continuous production process comprises the contacting. In some embodiments, the continuous production process comprises the saponifying. In some embodiments, the continuous production process comprises the acidifying.

In some embodiments, the method is a continuous process. In some embodiments, the reacting, distilling, contacting, saponifying, and/or acidifying is a continuous process. In some embodiments, the reacting and/or distilling is a continuous process. In some embodiments, the distilling is a continuous process. In some embodiments, the contacting is a continuous process. In some embodiments, the saponifying and/or acidifying is a continuous process. In some embodiments, the saponifying is a continuous process. In some embodiments, the acidifying is a continuous process.

In some embodiments, the method produces the one or more lactones in an amount of at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more, kg/h. In some embodiments, the method produces the one or more lactones in an amount of about 1000, 990, 980, 970, 960, 950, 940, 930, 920, 910, 900, 890, 880, 870, 860, 850, 840, 830, 820, 810, 800, 790, 780, 770, 760, 750, 740, 730, 720, 710, 700, 690, 680, 670, 660, 650, 640, 630, 620, 610, 600, 590, 580, 570, 560, 550, 540, 530, 520, 510, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, 10, or less, kg/h. In some instances, the method produces the one or more lactones in an amount of between about 10 and about 1000 kg/h.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Method with Saponification of Heavy Oil

Bio-derived lipids were saponified according to the following process. Heavy oil, a mixture of lipids described by Formula (I-A) and Formula (I-B), was produced via fermentation of A. pullulans in an aerobic environment utilizing xylose, glucose, sucrose, and arabinose among other carbon sources. The heavy oil product has a carbohydrate headgroup, and a chain of 1 to 5 O-acylated medium-chain fatty acids. The complete fermentation design includes feed holding tanks, fermentation broth holding tanks, process cooling, vent gas scrubber, and the CIP system. The heavy oil was purified via solvent extraction with methyl ethyl ketone and the solvent was removed through vacuum distillation and centrifugation.

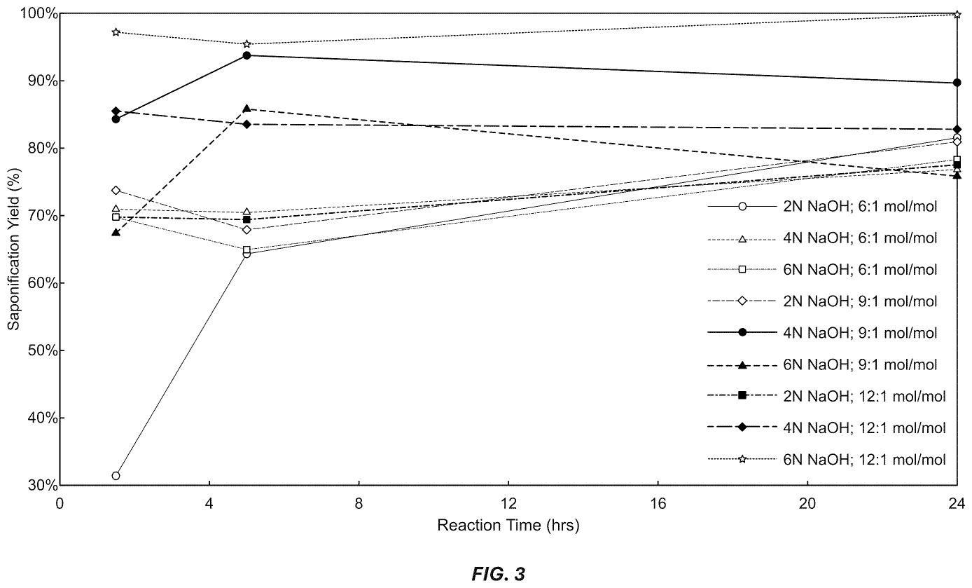

Dilute NaOH was then added to the purified heavy oil and the resultant mixture was agitated at 30° C. for up to 24 hours. The NaOH concentration, NaOH:oil molar ratio, and reaction time were all varied across multiple runs. Quantities of heavy oil and base were varied such that each run had 20 mL of saponification mixture (combined heavy oil and base). Following saponification, samples were brought to a neutral pH and filtered before HPLC analysis. Concentration of the heavy oil's carbohydrate headgroup, a product of the saponification reaction, was used to determine saponification yield. Depending on conditions, saponification yield varied from about 31% to about 100%. For example, when the heavy oil was saponified with 2N NaOH at an NaOH:oil ratio of 6:1 (mol/mol), the carbohydrate yield (as compared to the theoretical maximum yield) was 31% after 1.5 hours, 64% after 5 hours, and 82% after 24 hours of agitation at 30° C. Saponification yield for multiple runs is shown in Table 2 and FIG. 3.

The resultant saponified oil was used in the process described in Example 2. FIG. 3 depicts saponification yield vs time for various concentration and quantity of base.

TABLE 2

| | NaOH | | | | |
|---|---|---|---|---|---|
| Run | Concentration | NaOH:oil | 1.5 hr | 5 hr | 24 hr |
| # | (N) | (mol/mol) | yield (%) | yield (%) | yield (%) |
| 1 | 2 | 6 | 31% | 64% | 82% |
| 2 | 4 | 6 | 71% | 70% | 77% |
| 3 | 6 | 6 | 70% | 65% | 78% |
| 4 | 2 | 9 | 74% | 68% | 81% |
| 5 | 4 | 9 | 84% | 94% | 90% |
| 6 | 6 | 9 | 67% | 86% | 76% |
| 7 | 2 | 12 | 70% | 69% | 78% |
| 8 | 4 | 12 | 86% | 84% | 83% |
| 9 | 6 | 12 | 97% | 95% | 100% |

Table 2 depicts saponification yield vs time for various concentration an quantity of base.

### Example 2: Method of Preparing Lactones Via Acidification

Saponified heavy oil was prepared as described in Example 1. To this heavy oil, 5M H₂SO₄ was slowly added so as to not heat the oil mixture beyond 60° C., until the aqueous phase of the mixture reached a pH of about 2. Phase separation of the organic and aqueous phases occurred when the pH reached about 4. After the desired pH was reached, the mixture was stirred for at least one hour and then the aqueous phase was removed via gravity separation. Aqueous phases were analyzed and found to contain negligible lactone concentrations, and then disposed of Table B shows the concentrations of 5-hydroxy-2-decenoic acid-δ-lactone (Massoia lactone) and 3,5-dihydroxydecanoic acid-δ-lactone in the organic phase of the resultant acidified oil mixture for various pHs.

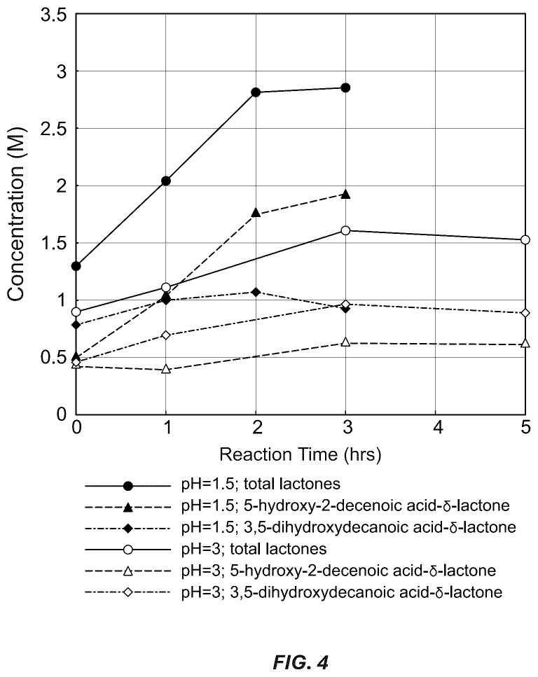

Following the addition of H₂SO₄, the organic phase was heated to 135° C. under reflux dewatering (e.g., with a Dean-Stark trap). Samples were taken hourly and analyzed via GC-MS to determine concentrations of 5-hydroxy-2-decenoic acid-δ-lactone (Massoia lactone) and 3,5-dihydroxydecanoic acid-δ-lactone. For a sample with a starting aqueous pH of 1.5, the total lactone concentration after 3 hours at 135° C. was about 2.9 M and the ratio of 5-hydroxy-2-decenoic acid-δ-lactone to 3,5-dihydroxydecanoic acid-δ-lactone was about 2.1. FIG. 4 shows lactone concentrations vs time held at 135° C. for oil mixtures with starting pHs of about 1.5 and about 3.

TABLE 3

| | | 5-hydroxy-2-decenoic | 3,5-dihydroxydecanoic | Total lactone |
|---|---|---|---|---|
| | | acid-δ-lactone | acid-δ-lactone | concentration |
| Sample # | pH | concentration (M) | concentration (M) | (M) |
| 1 | 1.6 | 0.91 | 0.29 | 1.20 |
| 2 | 1.155 | 0.68 | 0.26 | 0.94 |
| 3 | 0.99 | 0.70 | 0.29 | 0.99 |
| 4 | 0.35 | 0.57 | 0.29 | 0.86 |

Table 3 depicts initial concentrations of 5-hydroxy-2-decenoic acid-δ-lactone (Massoia lactone) and 3,5-dihydroxydecanoic acid-δ-lactone for various starting pHs.

FIG. 4 shows concentrations of 5-hydroxy-2-decenoic acid-δ-lactone (dashes), 3,5-dihydroxydecanoic acid-δ-lactone (dots), and total lactones (solid) versus reaction time for mixtures with a starting pH of 1.5 (black) and 3.0 (grey). Reaction was held at 135° C. under constant stirring and reflux dewatering.

Following acidification with H₂SO₄, the lactone-containing organic phase was brought to a neutral pH of about 7 by washing multiple times with NaHCO₃ at a 1:1 organic phase:NaHCO₃ mass ratio. Hexanoic acid, present in the acidified organic phase, was converted to hexanal during neutralization. The concentration of hexanoic acid, measured via GC-MS, was used to determine the extent of neutralization for successive NaHCO₃ washes. Each NaHCO₃ wash produced CO₂ and salt precipitants from the acid/base reaction. Table 4 shows hexanoic acid concentration and total sample mass for each successive NaHCO₃ wash.

TABLE 4

| | | % original hexanoic acid |
|---|---|---|
| Wash # | % original mass | concentration |
| 0 (acidified | 100% | 100% |
| organic phase) | | |
| 1 | 86% | 52% |
| 2 | 74% | 30% |
| 3 | 67% | 12% |

Table 4 depicts mass of lactone-containing sample and concentration of hexanoic acid, each as a percentage relative to pre-neutralization, for each successive 1:1 wash with NaHCO₃.

Following neutralization, samples were distilled via vacuum distillation at a temperature of about 140° C. and a pressure below 10 mbar in order to obtain a nominally pure lactone mixture.

### Example 3: Method of Preparing Lactone Mixture

Heavy oil was produced via fermentation of A. pullulans in an aerobic environment utilizing xylose, glucose, sucrose, and arabinose among other carbon sources. The heavy oil product has a mannitol headgroup, and a chain of three hydroxylated decanoic acid molecules. The complete fermentation design includes feed holding tanks, fermentation broth holding tanks, process cooling, vent gas scrubber, and the CIP system.

The resulting crude heavy oil was processed via the downstream processing block. The downstream processing block includes a Coflore liquid-liquid extraction unit, a water stripper distillation column, and a two liquid phase decanter system. In this system, fermentation broth is counter-currently exposed to MEK solvent, preferentially extracting the heavy oil product. An extract phase, saturated in water but enriched in the heavy oil was sent to the distillation block. The raffinate phase, deficient in the heavy oil, contained the cells and other water-soluble components. The raffinate was sent to the stripping column (reboiler operated at 100° C. and condenser operated at 73° C.), where any remaining MEK is stripped from the system before the water stream is discharged for wastewater treatment. Recycled MEK and water are mixed and allowed to settle in the decanter (operated at 40° C.). The aqueous and organic phases are immiscible, and are separated before being recycled to the stripper and liquid-liquid extraction units respectively.

The mass flow of the fermentation broth is about 17,693 kg/h and the solvent to fermentation broth ratio is about 0.5. The flow rate of the extract leaving this block is about 8705 kg/h and contains about 99.95 wt % of the heavy oil product.

Distillation Route A. The heavy oil product was saponified in sodium hydroxide. A 12:1 base:oil molar ratio was utilized. The saponification was completed at about 30° C. followed by lactonization in dilute mineral acid (e.g., sulfuric acid or hydrochloric acid at pH of about 2.5). The lactonization steps occurred at about 150° C. Following lactonization, the sample was neutralized with 9% NaHCO₃/H₂O for a total of three washes. This results in a crude lactone product which is about 50 wt % lactones, as measured by GC-MS.

The crude lactone product was then further purified via vacuum distillation, which took place at less than 200° C. under vacuum (pressure<10 mbar). The distillation resulted in the production of the lactone products, 5-hydroxy-2-decenoic acid-δ-lactone and 3,5-dihydroxydecanoic acid-δ-lactone.

Distillation Route B. The heavy oil product was acidified in concentrated mineral acid (e.g., sulfuric acid or hydrochloric acid) at a pH of about 0. The acidification took place at a temperature of about 150° C. This was followed by vacuum distillation at less than 200° C. under vacuum (pressure<10 mbar). The distillation resulted in the production of the lactone products, 5-hydroxy-2-decenoic acid-δ-lactone and 3,5-dihydroxydecanoic acid-6-lactone.

Distillation Route C. Thermal lactonization of the saponified heavy oil was completed at a temperature of greater than 250° C. in the absence of a base or an acid catalyst, followed by (or concurrently) distilling the lactone products, 5-hydroxy-2-decenoic acid-δ-lactone and 3,5-dihydroxydecanoic acid-δ-lactone.

In each distillation route, the resulting stream was sent to the fuel production block. The flow rate of the heavy oil to the distillation block is about 888 kg/h. The estimated conversion efficacy is about 1% conversion to residue, 4% conversion to water, 24% conversion to mannitol, and 71% conversion to lactones.

### ADDITIONAL EMBODIMENTS

The following exemplary embodiments are provided, the order of which is not to be construed as designating levels of importance:

Provided herein, in some embodiments, are compositions comprising (a) a 5-hydroxy-2-decenoic acid-δ-lactone; (b) a 3,5-dihydroxydecanoic acid-δ-lactone; and (c) sulfur, nitrogen, phosphorus, potassium, magnesium, calcium, sodium, iron, aluminum, zinc, manganese, copper, silicon, organic chloride, 3,5-dihydroxydecanoic acid, a carbohydrate, free fatty acids, or a combination thereof.

In some embodiments, a variety of one or more impurities may be present, such as 0.0 to 1.0 wt % hexanal, 0.0 to 1.0 wt % 4,4a,5,6,7,8a-h exahydropyrano[2,3-b]pyran, 0.0 to 1.0 wt % 2H,7H-pyrano[2,3-b]pyran, 0.0 to 5.0 wt % 2-methyl-3-heptyn-2-ol, 0.0 to 5.0 wt % 2,6-dimethyl-4-hepten-3-one, or combinations thereof.

In some embodiments, the 5-hydroxy-2-decenoic acid-δ-lactone is (R)-5,6-dihydro-6-pentyl-2H-pyran-2-one. In some embodiments, the compositions comprise water. In some embodiments, the compositions comprise at least 10 ppm sulfur. In some embodiments, the compositions comprise at least 37 ppm sulfur. In some embodiments, the compositions comprise at least 50 ppm nitrogen. In some embodiments, the compositions comprise at least 82 ppm nitrogen. In some embodiments, the compositions comprise at least 0.01 ppm phosphorus. In some embodiments, the compositions comprise at least 0.2 ppm phosphorus. In some embodiments, the compositions comprise at least 0.1 ppm potassium. In some embodiments, the compositions comprise at least 1.3 ppm potassium. In some embodiments, the compositions comprise at least 0.1 ppm magnesium. In some embodiments, the compositions comprise at least 1.3 ppm magnesium. In some embodiments, the compositions comprise at least 1 ppm calcium. In some embodiments, the compositions comprise at least 3.1 ppm calcium. In some embodiments, the compositions comprise at least 1 ppm sodium. In some embodiments, the compositions comprise at least 5.4 ppm sodium. In some embodiments, the compositions comprise at least 0.01 ppm iron. In some embodiments, the compositions comprise at least 0.2 ppm iron. In some embodiments, the compositions comprise at least 0.01 ppm aluminum. In some embodiments, the compositions comprise at least 0.3 ppm aluminum. In some embodiments, the compositions comprise at least 1 ppm zinc. In some embodiments, the compositions comprise at least 3 ppm zinc. In some embodiments, the compositions comprise at least 1 ppm manganese. In some embodiments, the compositions comprise at least 3 ppm manganese. In some embodiments, the compositions comprise at least 1 ppm copper. In some embodiments, the compositions comprise at least 3 ppm copper. In some embodiments, the compositions comprise at least 1 ppm silicon. In some embodiments, the compositions comprise at least 3.3 ppm silicon. In some embodiments, the compositions comprise at least 1 ppm organic chloride. In some embodiments, the compositions comprise the carbohydrate comprises mannitol. In some embodiments, the free fatty acids comprise C22:0 (e.g., behenic acid), C18:3 (e.g., linolenic acid), C24:0 (e.g., lignoceric acid), C18:1 (e.g., oleic acid), or C14:0 (e.g., myristic acid). In some embodiments, the free fatty acids comprise at least 50% C22:0, at least 20% C18:3, at least 10% C24:0, at least 1% C18:1, or at least 0.1% C14:0. In some embodiments, the free fatty acids comprise at least 52% C22:0, at least 21% C18:3, at least 17% C24:0, at least 5% C18:1, or at least 2% C14:0. In some embodiments, the compositions comprise cell debris. In some embodiments, the cell debris comprises A. pullulans cell debris.

In some embodiments, a variety of one or more impurities may be present, such as 0.0 to 1.0 wt % hexanal, 0.0 to 1.0 wt % 4,4a,5,6,7,8a-h exahydropyrano[2,3-b]pyran, 0.0 to 1.0 wt % 2H,7H-pyrano[2,3-b]pyran, 0.0 to 5.0 wt % 2-methyl-3-heptyn-2-ol, 0.0 to 5.0 wt % 2,6-dimethyl-4-hepten-3-one, or combinations thereof.

Provided herein, in some embodiments, are food products comprising the compositions described herein. Provided herein, in some embodiments, are cosmetic products comprising the compositions described herein. Provided herein, in some embodiments, are antifungal products comprising the compositions described herein.

Provided herein, in some embodiments, are methods comprising (a) providing a bioproduct comprising one or more lipids, the one or more lipids comprising a carbohydrate and 1 to 5 C₈-C₁₆ polyester tails. In some embodiments, the methods comprise (b) distilling the bioproduct, wherein the distilling produces a composition comprising one or more substituted or unsubstituted lactones.

In some embodiments, the methods comprise saponifying the bioproduct with a base. In some embodiments, the saponifying is a continuous process. In some embodiments, the methods comprise contacting at least a portion of the one or more lipids with a base. In some embodiments, the contacting is a continuous process. In some embodiments, the methods comprise acidifying the bioproduct with a catalyst. In some embodiments, the acidifying is a continuous process. In some embodiments, the methods comprise contacting at least a portion of the one or more lipids with a catalyst. In some embodiments, the contacting is a continuous process.

Provided herein, in some embodiments, are methods comprising (a) providing a bioproduct comprising one or more lipids, the one or more lipids comprising a carbohydrate and 1 to 5 C₈-C₁₆ polyester tails. In some embodiments, the methods comprise (b) reacting and distilling the bioproduct, wherein the distilling produces a composition comprising one or more substituted or unsubstituted lactones. In some embodiments, the methods comprise a combination thereof.

In some embodiments, the reacting comprises (i) contacting at least a portion of the one or more lipids with a base or (ii) contacting at least a portion of the one or more lipids with a catalyst. In some embodiments, the reacting is a continuous process.

Provided herein, in some embodiments, are methods comprising (a) providing a bioproduct comprising one or more lipids, the one or more lipids comprising a carbohydrate and 1 to 5 C₈-C₁₆ polyester tails. In some embodiments, the methods comprise (b) either (i) contacting at least a portion of the one or more lipids with a base or (ii) contacting at least a portion of the one or more lipids with a catalyst. In some embodiments, the methods comprise (c) distilling the bioproduct, wherein the distilling produces a composition comprising one or more substituted or unsubstituted lactones. In some embodiments, the methods comprise a combination thereof. In some embodiments, the contacting is a continuous process.

Provided herein, in some embodiments, are methods comprising (a) harvesting a bioproduct produced by an engineered organism, the bioproduct comprising one or more lipids comprising a carbohydrate and 1 to 5 C₈-C₁₆ polyester tails polyester tails. In some embodiments, the methods comprise (b) distilling the bioproduct, wherein the distilling produces a composition comprising one or more substituted or unsubstituted lactones.

In some embodiments, the methods comprise saponifying the bioproduct with a base. In some embodiments, the saponifying is a continuous process. In some embodiments, the methods comprise contacting at least a portion of the one or more lipids with a base. In some embodiments, the methods comprise acidifying the bioproduct with a catalyst. In some embodiments, the acidifying is a continuous process. In some embodiments, the methods comprise contacting at least a portion of the one or more lipids with a catalyst. In some embodiments, the methods comprise the contacting is a continuous process.

Provided herein, in some embodiments, are methods comprising (a) harvesting a bioproduct produced by an engineered organism, the bioproduct comprising one or more lipids comprising a carbohydrate and 1 to 5 C₈-C₁₆ polyester tails. In some embodiments, the methods comprise (b) reacting and distilling the bioproduct, wherein the distilling produces a composition comprising one or more substituted or unsubstituted lactones. In some embodiments, the methods comprise a combination thereof.

In some embodiments, the reacting comprises (i) contacting at least a portion of the one or more lipids with a base or (ii) contacting at least a portion of the one or more lipids with a catalyst. In some embodiments, the reacting is a continuous process.

Provided herein, in some embodiments, are methods comprising (a) harvesting a bioproduct produced by an engineered organism, the bioproduct comprising one or more lipids comprising a carbohydrate and 1 to 5 C₈-C₁₆ polyester tails. In some embodiments, the methods comprise (b) either (i) contacting at least a portion of the one or more lipids with a base or (ii) contacting at least a portion of the one or more lipids with a catalyst. In some embodiments, the methods comprise (c) distilling the bioproduct, wherein the distilling produces a composition comprising one or more substituted or unsubstituted lactones. In some embodiments, the methods comprise a combination thereof.

In some embodiments, the contacting is a continuous process. In some embodiments, the one or more substituted or unsubstituted lactones comprise a 5-hydroxy-2-decenoic acid-δ-lactone, a 3,5-dihydroxydecanoic acid-δ-lactone, or a combination thereof. In some embodiments, wherein the distilling provides a yield of the one or more substituted or unsubstituted lactones of at least 70 wt %. In some embodiments, the base is potassium hydroxide, sodium hydroxide, sodium bicarbonate, or calcium hydroxide. In some embodiments, the contacting is completed at a temperature of at least 40° C.

In some embodiments, the catalyst is an acid catalyst. In some embodiments, the acid catalyst is hydrochloric acid, sulfuric acid, hydrobromic acid, phosphoric acid, iodic acid, or a combination thereof. In some embodiments, the acid catalyst has a pKₐ of about 0. In some embodiments, the acid catalyst has a pKₐ of 3 or less. In some embodiments, the acid catalyst has a pkₐ of about −1. In some embodiments, the contacting at least a portion of the one or more lipids with the catalyst is completed at a temperature of at least 100° C.

In some embodiments, the distilling comprises simple distillation, fractional distillation, vacuum distillation, azeotropic distillation, wiped film distillation, spinning band distillation, or a combination thereof. In some embodiments, the distilling is completed at a temperature of less than 200° C. In some embodiments, the distilling is completed at a temperature of at least 200° C. In some embodiments, the distilling is completed at a temperature of from 200° C. to 250° C. In some embodiments, the distilling is completed at a temperature of at least 250° C. In some embodiments, the distilling is completed at a temperature of from 100° C. to 150° C. In some embodiments, the distilling is completed under vacuum conditions.

In some embodiments, when the method comprises the saponifying step or the contacting with a base step, the distilling is completed at a temperature of at least 250° C. In some embodiments, when the method does not comprise the reacting, saponifying, acidifying, or contacting steps, the distilling is completed at a temperature of at least 250° C. In some embodiments, when the method comprises the reacting, saponifying, acidifying, or contacting steps, the distilling is completed at a temperature of less than 200° C.

In some embodiments, the engineered organism is of the genus Aureobasidium. In some embodiments, the engineered organism is of the species A. pullulans, A. melanogenum, or A. thailandense. In some embodiments, the engineered organism is A. pullulans. In some embodiments, the engineered organism comprises a modification to a gene related to polymalic acid, pullulan production, oxygen binding hemoglobin, a pathway activator gene, or a combination thereof.

In some embodiments, the methods comprise collecting the one or more lipids from the bioproduct via liquid-liquid extraction with a solvent. In some embodiments, the liquid-liquid extraction is a continuous process.

Provided herein, in some embodiments, are methods comprising (a) harvesting a bioproduct produced by an engineered organism, the bioproduct comprising one or more lipids comprising a carbohydrate and 1 to 5 C₈-C₁₆ polyester tails. In some embodiments, the methods comprise (b) contacting at least a portion of the one or more lipids with a base. In some embodiments, the methods comprise (c) distilling the bioproduct, wherein the distilling produces a composition comprising one or more substituted or unsubstituted lactones. In some embodiments, the methods comprise a combination thereof.

Provided herein, in some embodiments, are methods comprising (a) harvesting a bioproduct produced by an engineered organism, the bioproduct comprising one or more lipids comprising a carbohydrate and 1 to 5 C₈-C₁₆ polyester tails. In some embodiments, the methods comprise (b) contacting at least a portion of the one or more lipids with an acid catalyst. In some embodiments, the methods comprise (c) distilling the bioproduct, wherein the distilling produces a composition comprising one or more substituted or unsubstituted lactones. In some embodiments, the methods comprise a combination thereof.

Provided herein, in some embodiments, are methods comprising (a) harvesting a bioproduct produced by an engineered organism, the bioproduct comprising one or more lipids comprising a carbohydrate and 1 to 5 C₈-C₁₆ polyester tails. In some embodiments, the methods comprise (b) contacting at least a portion of the one or more lipids with a base. In some embodiments, the methods comprise (c) contacting at least a portion of the one or more lipids with an acid catalyst. In some embodiments, the methods comprise (d) distilling the bioproduct, wherein the distilling produces a composition comprising one or more substituted or unsubstituted lactones. In some embodiments, the methods comprise a combination thereof.

In some embodiments, the one or more lipids are represented by a structure of Formula (I):

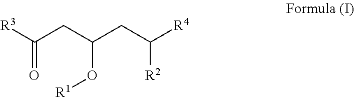

wherein,
R¹ is hydrogen (H) or an acyl group (CH₃CO);
R² is 1 to 10 O-acylated 3,5-dihydroxydecanoate and/or 3,5-dihydroxydodecanoate groups;
R³ is L- or D-glycerol, L- or D-threitol, L- or D-erythritol, L- or D-arabitol, L- or D-xylitol, L- or D-lyxitol, L- or D-ribitol, L- or D-allitol, L- or D-altritol, L- or D-mannitol, L- or D-iditol, L- or D-gulitol, L- or D-glucitol, L- or D-galactitol, L- or D-talitol, 2-amino-D-mannitol, 2N-acetylamino-D-mannitol, L-rhamnitol, or D-fucitol; and
R⁴ is C₅-C₇ alkyl.

In some embodiments, R² is 1 to 3 O-acylated 3,5-dihydroxydecanoate. In some embodiments, R⁴ is C₅ alkyl or C₇ alkyl. In some embodiments, R¹ is L- or D-mannitol, L- or D-glycerol, or L- or D-arabitol. In some embodiments, the one or more lipids comprise:

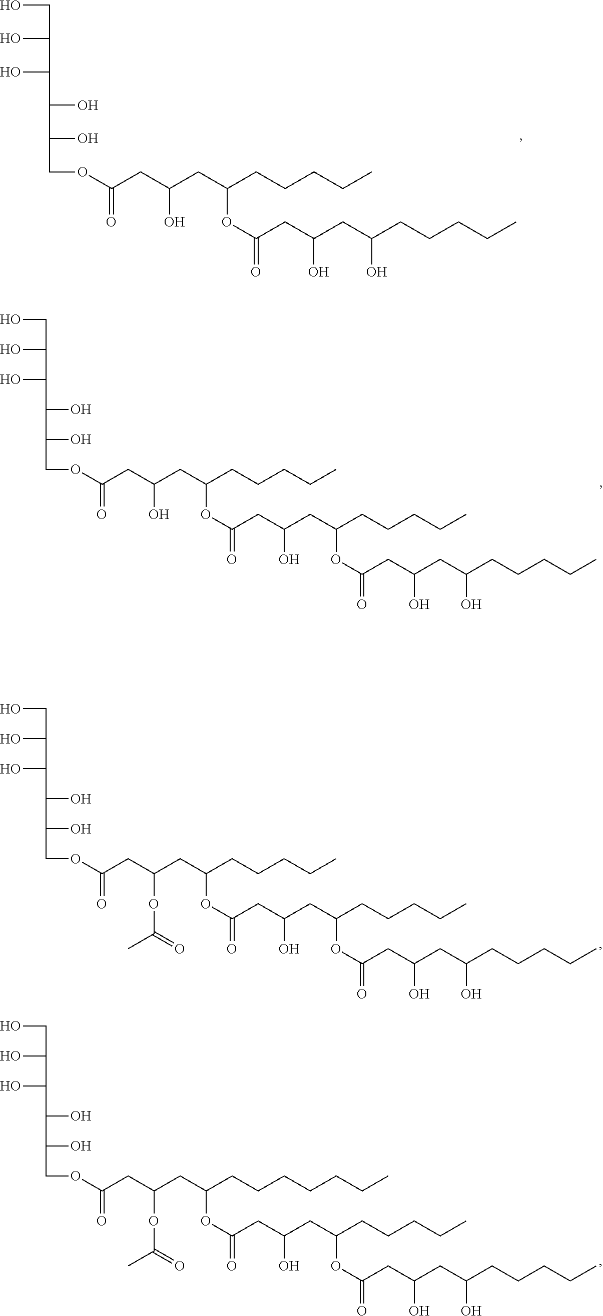 [SMILES: CCCCCC(O)CC(O)CC(=O)OC(CCCCC)CC(O)CC(=O)OCC(O)C(O)C(O)C(O)CO ; CCCCCC(O)CC(O)CC(=O)OC(CCCCC)CC(O)CC(=O)OC(CCCCC)CC(O)CC(=O)OCC(O)C(O)C(O)C(O)CO ; CCCCCC(O)CC(O)CC(=O)OC(CCCCC)CC(O)CC(=O)OC(CCCCC)CC(CC(=O)OCC(O)C(O)C(O)C(O)CO)OC(C)=O ; CCCCCCCC(CC(CC(=O)OCC(O)C(O)C(O)C(O)CO)OC(C)=O)OC(=O)CC(O)CC(CCCCC)OC(=O)CC(O)CC(O)CCCCC]

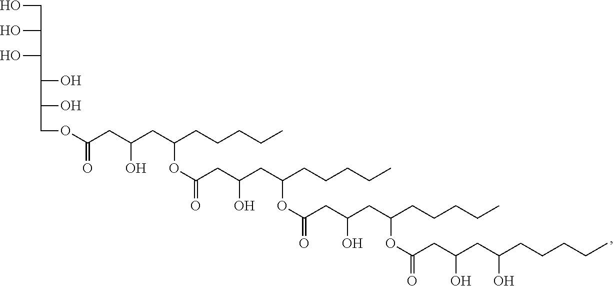 [SMILES: CCCCCC(O)CC(O)CC(=O)OC(CCCCC)CC(O)CC(=O)OC(CCCCC)CC(O)CC(=O)OC(CCCCC)CC(O)CC(=O)OCC(O)C(O)C(O)C(O)CO]

or a combination thereof.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

We claim:

1. A composition comprising:
(a) a 5-hydroxy-2-decenoic acid-δ-lactone;
(b) a 3,5-dihydroxydecanoic acid-δ-lactone; and
(c) sulfur, nitrogen, phosphorus, potassium, magnesium, calcium, sodium, iron, aluminum, zinc, manganese, copper, silicon, organic chloride, 3,5-dihydroxydecanoic acid, a carbohydrate, free fatty acids, or a combination thereof.

2. The composition of claim 1, wherein the 5-hydroxy-2-decenoic acid-δ-lactone is (R)-5,6-dihydro-6-pentyl-2H-pyran-2-one.

3. The composition of claim 1, comprising water.

4. The composition of claim 1, comprising at least 1 ppm sulfur, at least 1 ppm nitrogen, at least 0.01 ppm phosphorus, at least 0.1 ppm potassium, at least 0.1 ppm magnesium, at least 1 ppm calcium, at least 1 ppm sodium, at least 0.01 ppm iron, at least 0.01 ppm aluminum, at least 1 ppm zinc, at least 1 ppm manganese, at least 1 ppm copper, at least 1 ppm silicon, at least 1 ppm organic chloride, or combinations thereof.

5. The composition of claim 1, wherein the carbohydrate comprises mannitol, L- or D-glycerol, L- or D-threitol, L- or D-erythritol, L- or D-arabitol, L- or D-xylitol, L- or D-lyxitol, L- or D-ribitol, L- or D-allitol, L- or D-altritol, L- or D-mannitol, L- or D-iditol, L- or D-gulitol, L- or D-glucitol, L- or D-galactitol, L- or D-talitol, 2-amino-D-mannitol, 2N-acetylamino-D-mannitol, L-rhamnitol, D-fucitol, or combinations thereof.

6. The composition of claim 1, wherein the free fatty acids comprise C22:0 (e.g., behenic acid), C18:3 (e.g., linolenic acid), C24:0 (e.g., lignoceric acid), C18:1 (e.g., oleic acid), or C14:0 (e.g., myristic acid).

7. The composition of claim 1, wherein the free fatty acids comprise at least 50% C22:0, at least 20% C18:3, at least 10% C24:0, at least 1% C18:1, or at least 0.1% C14:0.

8. The composition of claim 1, wherein the free fatty acids comprise at least 52% C22:0, at least 21% C18:3, at least 17% C24:0, at least 5% C18:1, or at least 2% C14:0.

9. The composition of claim 1, comprising cell debris including A. pullulans cell debris.

10. A method comprising:
(a) harvesting a bioproduct produced by an engineered organism, the bioproduct comprising one or more lipids, the one or more lipids comprising a carbohydrate and 1 to 5 C₈-C₁₆ polyester tails polyester tails; and
(b) distilling the bioproduct, wherein the distilling produces a composition comprising one or more substituted or unsubstituted lactones.

11. The method of claim 10, further comprising either (i) contacting at least a portion of the one or more lipids with a base or (ii) contacting at least a portion of the one or more lipids with a catalyst.

12. The method of claim 10, wherein the engineered organism is of the genus Aureobasidium.

13. The method of claim 12, wherein the engineered organism is of the species A. pullulans, A. melanogenum, or A. thailandense.

14. The method of claim 10, wherein the engineered organism comprises a modification to a gene related to polymalic acid, pullulan production, oxygen binding hemoglobin, a pathway activator gene, or a combination thereof.

15. A method comprising:
(a) Harvesting a bioproduct produced by an engineered organism, the bioproduct comprising one or more lipids, the one or more lipids comprising a carbohydrate and 1 to 5 C₈-C₁₆ polyester tails;
(b) contacting at least a portion of the one or more lipids with a base or with an acid catalyst; and
(c) distilling the bioproduct, wherein the distilling produces a composition comprising one or more substituted or unsubstituted lactones, or a combination thereof.

16. The method of claim 15, wherein the one or more lipids are represented by a structure of Formula (I): 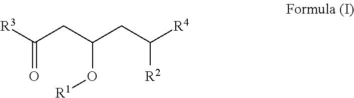
wherein,
R¹ is hydrogen (H) or an acyl group (CH₃CO);
R² is 1 to 10 O-acylated 3,5-dihydroxydecanoate; and/or 3,5-dihydroxydodecanoate groups;
R³ is L- or D-glycerol, L- or D-threitol, L- or D-erythritol, L- or D-arabitol, L- or D-xylitol, L- or D-lyxitol, L- or D-ribitol, L- or D-allitol, L- or D-altritol, L- or D-mannitol, L- or D-iditol, L- or D-gulitol, L- or D-glucitol, L- or D-galactitol, L- or D-talitol, 2-amino-D-mannitol, 2N-acetylamino-D-mannitol, L-rhamnitol, or D-fucitol; and
R⁴ is C₅-C₇ alkyl.

17. The method of claim 15, wherein R² is 1 to 3 O-acylated 3,5-dihydroxydecanoate.

18. The method of claim 15, wherein R⁴ is C₅ alkyl or C₇ alkyl.

19. The method of claim 15, wherein R¹ is L- or D-mannitol, L- or D-glycerol, or L- or D-arabitol.

20. The method of claim 15, wherein the one or more lipids comprise: 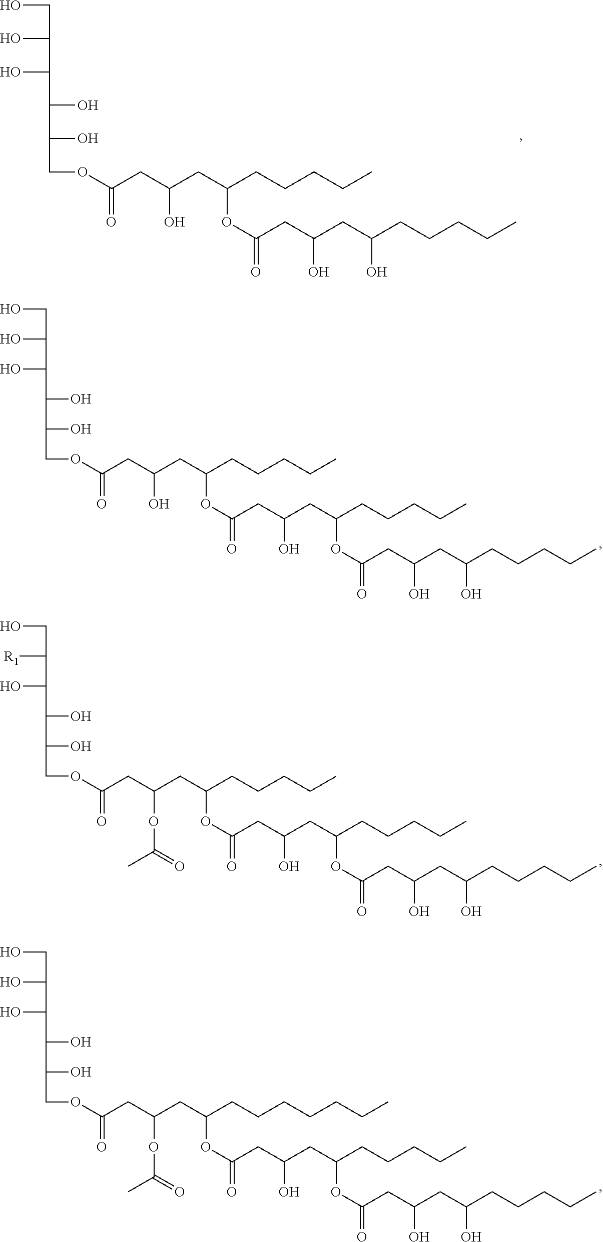 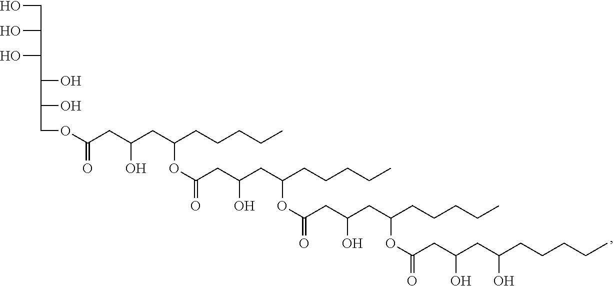 [SMILES: CCCCCC(O)CC(O)CC(=O)OC(CCCCC)CC(O)CC(=O)OC(CCCCC)CC(O)CC(=O)OC(CCCCC)CC(O)CC(=O)OCC(O)C(O)C(O)C(O)CO]
or a combination thereof.
